SEQUENCE LISTING (62 sequences)

SEQ ID NO: 1 - DNA, 38 bp, Artificial Sequence
  Synthetic oligonucleotide
  misc_feature (1)..(1): FAM on 5′ end
  misc_feature (2)..(2): n is inosine
  misc_feature (4)..(4): n is inosine
  misc_feature (7)..(7): n is inosine
  misc_feature (11)..(11): n is inosine
  misc_feature (14)..(14): n is inosine
  misc_feature (17)..(18): n is inosine
  misc_feature (22)..(23): n is inosine
  misc_feature (27)..(27): n is inosine
  misc_feature (30)..(30): n is inosine
  misc_feature (32)..(33): n is inosine
  misc_feature (36)..(37): n is inosine
  misc_feature (38)..(38): Cy5 on 3′ end
nntnttntttnttnttnntttnntttnttntnnttnnn

SEQ ID NO: 2 - DNA, 38 bp, Artificial Sequence
  Synthetic oligonucleotide
  misc_feature (1)..(1): IBRQ at 5′ end
  misc_feature (38)..(38): IBFQ at 3′ end
cccaaccacaacaaaccaaaccaacaacaaacaacacc

SEQ ID NO: 3 - DNA, 38 bp, Artificial Sequence
  Synthetic oligonucleotide
ggtgttgtttgttgttggtttggtttgttgtggttggg

SEQ ID NO: 4 - DNA, 38 bp, Artificial Sequence
  Synthetic oligonucleotide
gatgttatttgttattggtttagtttgttatggttagg

SEQ ID NO: 5 - DNA, 38 bp, Artificial Sequence
  Synthetic oligonucleotide
aatattgtttattgttagtttgatttgttatgattgag

SEQ ID NO: 6 - DNA, 38 bp, Artificial Sequence
  Synthetic oligonucleotide
gatgttatttgttattgatttagtttattgtgattgag

SEQ ID NO: 7 - DNA, 38 bp, Artificial Sequence
  Synthetic oligonucleotide
gttgttttttgttgttgtttttgttttttgttgttgtg

SEQ ID NO: 8 - DNA, 38 bp, Artificial Sequence
  Synthetic oligonucleotide
tttgttttttgtttttggttttgtttgttttgtttgtt

SEQ ID NO: 9 - DNA, 38 bp, Artificial Sequence
  Synthetic oligonucleotide
ttgtggtgggtggtggttgggttgggtggtgttggttt

SEQ ID NO: 10 - DNA, 54 bp, Artificial Sequence
  Synthetic oligonucleotide
  misc_feature (1)..(1): Cy5.5 at 5′ end
  misc_feature (2)..(4): n is inosine
  misc_feature (7)..(8): n is inosine
  misc_feature (10)..(10): n is inosine
  misc_feature (13)..(13): n is inosine
  misc_feature (17)..(18): n is inosine
tnnnttnntnttntttnntttcttctttcttcttgaccagtcgcatggatcggc

SEQ ID NO: 11 - DNA, 54 bp, Artificial Sequence
  Synthetic oligonucleotide
  misc_feature (54)..(54): IBRQ at 3′ end
gccgatccatgcgactggtcatttccctctcccaaaccaaacaacaccaaccca

SEQ ID NO: 12 - DNA, 28 bp, Artificial Sequence
  Synthetic oligonucleotide
aggagaatgggttggtgttgtttggttt

SEQ ID NO: 13 - DNA, 21 bp, Artificial Sequence
  Synthetic oligonucleotide
acaacaccaacccattctcct

SEQ ID NO: 14 - DNA, 21 bp, Artificial Sequence
  Synthetic oligonucleotide
acaacaccaacccattctcct

SEQ ID NO: 15 - DNA, 32 bp, Artificial Sequence
  Synthetic oligonucleotide
gtgttgtttggtttgggagagggtctcctttc

SEQ ID NO: 16 - DNA, 32 bp, Artificial Sequence
  Synthetic oligonucleotide
gaaaggagaccctctcccaaaccaaacaacac

SEQ ID NO: 17 - DNA, 24 bp, Artificial Sequence
  Synthetic oligonucleotide
gtgttgtttggtttgggagaggga

SEQ ID NO: 18 - DNA, 24 bp, Artificial Sequence
  Synthetic oligonucleotide
  misc_feature (1)..(1): FAM at 5′ end
gtgttgtttggtttgggagaggga

SEQ ID NO: 19 - DNA, 24 bp, Artificial Sequence
  Synthetic oligonucleotide
tccctctcccaaaccaaacaacac

SEQ ID NO: 20 - DNA, 24 bp, Artificial Sequence
  Synthetic oligonucleotide
tccctctcccaaaccaaacaacac

SEQ ID NO: 21 - DNA, 24 bp, Artificial Sequence
  Synthetic oligonucleotide
tccctctcccaaaccaaacaacac

SEQ ID NO: 22 - DNA, 21 bp, Artificial Sequence
  Synthetic oligonucleotide
tgggttggtgttgtttggttt

SEQ ID NO: 23 - DNA, 21 bp, Artificial Sequence
  Synthetic oligonucleotide
aaaccaaacaacaccaaccca

SEQ ID NO: 24 - DNA, 96 bp, Artificial Sequence
  Synthetic oligonucleotide
  misc_feature (27)..(28): n is inosine
  misc_feature (30)..(30): n is inosine
  misc_feature (33)..(33): n is inosine
  misc_feature (37)..(37): n is inosine
  misc_feature (40)..(40): n is inosine
  misc_feature (43)..(44): n is inosine
  misc_feature (48)..(49): n is inosine
  misc_feature (53)..(53): n is inosine
  misc_feature (56)..(56): n is inosine
  misc_feature (58)..(59): n is inosine
  misc_feature (62)..(64): n is inosine
gccatagttagagcatgcgccatagtnntnttntttnttnttnntttnntttnttntnnttnnntcttttccgaatgcagctgccattccgaatgc

SEQ ID NO: 25 - DNA, 96 bp, Artificial Sequence
  Synthetic oligonucleotide
cgcaatccaccgatcatccgcaatccaaatctcccaaccacaacaaaccaaaccaacaacaaacaacaccactatggcgcatgctctaactatggc

SEQ ID NO: 26 - DNA, 96 bp, Artificial Sequence
  Synthetic oligonucleotide
gccatagttagagcatgcgccatagtggtgttgtttgttgttggtttggtttgttgtggttgggtcttttccgaatgcagctgccattccgaatgc

SEQ ID NO: 27 - DNA, 26 bp, Artificial Sequence
  Synthetic oligonucleotide
  misc_feature (26)..(26): FAM at 3′ end
gcattcggaatggcagctgcattcgg

SEQ ID NO: 28 - DNA, 32 bp, Artificial Sequence
  Synthetic oligonucleotide
  misc_feature (32)..(32): FAM at 3′ end
gcattcggaatggcagctgcattcggaaaaga

SEQ ID NO: 29 - DNA, 26 bp, Artificial Sequence
  Synthetic oligonucleotide
  misc_feature (1)..(1): IbFQ at 5′ end
ggattgcggatgatcggtggattgcg

SEQ ID NO: 30 - DNA, 26 bp, Artificial Sequence
  Synthetic oligonucleotide
  misc_feature (1)..(1): Cy3 at 5′ end
  misc_feature (2)..(2): n is inosine
  misc_feature (6)..(6): n is inosine
  misc_feature (8)..(9): n is inosine
  misc_feature (12)..(12): n is inosine
  misc_feature (16)..(17): n is inosine
  misc_feature (19)..(20): n is inosine
  misc_feature (24)..(24): n is inosine
  misc_feature (26)..(26): Cy5 at 3′ end
nnattncnnatnatcnntnnattncn

SEQ ID NO: 31 - DNA, 26 bp, Artificial Sequence
  Synthetic oligonucleotide
ggattgcggatgatcggtggattgcg

SEQ ID NO: 32 - DNA, 20 bp, Artificial Sequence
  Synthetic oligonucleotide
  misc_feature (1)..(1): IbFQ at 5′ end
cggatgatcggtggattgcg

SEQ ID NO: 33 - DNA, 62 bp, Artificial Sequence
  Synthetic oligonucleotide
agaagtaagtagggtgttgtttgttgttggtttggtttgttgtggttgggaagtgagcgtaa

SEQ ID NO: 34 - DNA, 62 bp, Artificial Sequence; identical to SEQ ID NO: 33
  Synthetic oligonucleotide
  misc_feature (1)..(1): 5Cy5 at 5′ end

SEQ ID NO: 35 - DNA, 27 bp, Artificial Sequence
  Synthetic oligonucleotide
acaacaaacaacaccctacttacttct

SEQ ID NO: 36 - DNA, 27 bp, Artificial Sequence
  Synthetic oligonucleotide
  misc_feature (27)..(27): 3IbRQ at 3′ end
acaacaaacaacaccctacttacttct

SEQ ID NO: 37 - DNA, 27 bp, Artificial Sequence
  Synthetic oligonucleotide
ttacgctcacttcccaaccacaacaaa

SEQ ID NO: 38 - DNA, 56 bp, Artificial Sequence
  Synthetic oligonucleotide
ggtgttgtttgttgttggtttggtttgttgtggttgggagatttaagtgagcgtaa

SEQ ID NO: 39 - DNA, 36 bp, Artificial Sequence
  Synthetic oligonucleotide
  misc_feature (1)..(1): 5IAbFQ at 5′ end
ttacgctcacttaaatctcccaaccacaacaaacca

SEQ ID NO: 40 - DNA, 36 bp, Artificial Sequence; identical to SEQ ID NO: 39
  Synthetic oligonucleotide

SEQ ID NO: 41 - DNA, 56 bp, Artificial Sequence; identical to SEQ ID NO: 38
  Synthetic oligonucleotide
  misc_feature (56)..(56): 36-FAM at 3′ end

SEQ ID NO: 42 - DNA, 68 bp, Artificial Sequence
  Synthetic oligonucleotide
agaagtaagtagggtgttgtttgttgttggtttggtttgttgtggttgggagatttaagtgagcgtaa

SEQ ID NO: 43 - DNA, 33 bp, Artificial Sequence
  Synthetic oligonucleotide
ttacgctcacttaaatctcccaaccacaacaaa

SEQ ID NO: 44 - DNA, 74 bp, Artificial Sequence
  Synthetic oligonucleotide
agaagtaagtagggtgttgtttgttgttggtttggtttgttgtggttgggagatttggattgaagtgagcgtaa

SEQ ID NO: 45 - DNA, 42 bp, Artificial Sequence
  Synthetic oligonucleotide
ttacgctcacttcaatccaaatctcccaaccacaacaaacca

SEQ ID NO: 46 - DNA, 39 bp, Artificial Sequence
  Synthetic oligonucleotide
ttacgctcacttcaatccaaatctcccaaccacaacaaa

SEQ ID NO: 47 - DNA, 38 bp, Artificial Sequence; identical to SEQ ID NO: 3
  Synthetic oligonucleotide

SEQ ID NO: 48 - DNA, 38 bp, Artificial Sequence
  Synthetic oligonucleotide
  misc_feature (1)..(1): n is inosine
  misc_feature (11)..(11): n is inosine
  misc_feature (22)..(22): n is inosine
  misc_feature (27)..(27): n is inosine
  misc_feature (37)..(37): n is inosine
ngtgttgtttnttgttggtttngtttnttgtggttgng

SEQ ID NO: 49 - DNA, 38 bp, Artificial Sequence
  Synthetic oligonucleotide
  misc_feature (1)..(1): n is inosine
  misc_feature (7)..(7): n is inosine
  misc_feature (14)..(14): n is inosine
  misc_feature (22)..(22): n is inosine
  misc_feature (30)..(30): n is inosine
  misc_feature (36)..(37): n is inosine
ngtgttntttgttnttggtttngtttgttntggttnng

SEQ ID NO: 50 - DNA, 38 bp, Artificial Sequence
  Synthetic oligonucleotide
  misc_feature (1)..(1): n is inosine
  misc_feature (4)..(4): n is inosine
  misc_feature (11)..(11): n is inosine
  misc_feature (17)..(17): n is inosine
  misc_feature (22)..(22): n is inosine
  misc_feature (27)..(27): n is inosine
  misc_feature (32)..(32): n is inosine
  misc_feature (36)..(36): n is inosine
  misc_feature (38)..(38): n is inosine
ngtnttgtttnttgttngtttngtttnttgtngttngn

SEQ ID NO: 51 - DNA, 44 bp, Artificial Sequence
  Synthetic oligonucleotide
  misc_feature (2)..(2): n is inosine
  misc_feature (4)..(4): n is inosine
  misc_feature (7)..(7): n is inosine
  misc_feature (14)..(14): n is inosine
  misc_feature (17)..(18): n is inosine
  misc_feature (23)..(23): n is inosine
  misc_feature (27)..(27): n is inosine
  misc_feature (30)..(30): n is inosine
  misc_feature (32)..(33): n is inosine
  misc_feature (36)..(36): n is inosine
  misc_feature (38)..(38): n is inosine
gntnttntttgttnttnntttgntttnttntnnttngnaagtga

SEQ ID NO: 52 - DNA, 38 bp, Artificial Sequence; identical to SEQ ID NO: 1
  Synthetic oligonucleotide
  misc_feature (1)..(2): n is inosine
  misc_feature (4)..(4): n is inosine
  misc_feature (7)..(7): n is inosine
  misc_feature (11)..(11): n is inosine
  misc_feature (14)..(14): n is inosine
  misc_feature (17)..(18): n is inosine
  misc_feature (22)..(23): n is inosine
  misc_feature (27)..(27): n is inosine
  misc_feature (30)..(30): n is inosine
  misc_feature (32)..(33): n is inosine
  misc_feature (36)..(38): n is inosine

SEQ ID NO: 53 - DNA, 38 bp, Artificial Sequence; identical to SEQ ID NO: 1
  Synthetic oligonucleotide
  misc_feature (1)..(1): FAM at 5′ end
  misc_feature (2)..(2): n is inosine
  misc_feature (4)..(4): n is inosine
  misc_feature (7)..(7): n is inosine
  misc_feature (11)..(11): n is inosine
  misc_feature (14)..(14): n is inosine
  misc_feature (17)..(18): n is inosine
  misc_feature (22)..(23): n is inosine
  misc_feature (27)..(27): n is inosine
  misc_feature (30)..(30): n is inosine
  misc_feature (32)..(33): n is inosine
  misc_feature (36)..(37): n is inosine
  misc_feature (38)..(38): Cy5 at 3′ end

SEQ ID NO: 54 - DNA, 38 bp, Artificial Sequence; identical to SEQ ID NO: 2
  Synthetic oligonucleotide
  misc_feature (1)..(1): IbRQ at 5′ end
  misc_feature (38)..(38): IbFQ at 3′ end

SEQ ID NO: 55 - DNA, 38 bp, Artificial Sequence; identical to SEQ ID NO: 3
  Synthetic oligonucleotide

SEQ ID NO: 56 - DNA, 26 bp, Artificial Sequence
  Synthetic oligonucleotide
  misc_feature (1)..(1): Cy3 at 5′ end
  misc_feature (2)..(2): n is inosine
  misc_feature (6)..(6): n is inosine
  misc_feature (8)..(9): n is inosine
  misc_feature (12)..(12): n is inosine
  misc_feature (16)..(17): n is inosine
  misc_feature (19)..(20): n is inosine
  misc_feature (24)..(24): n is inosine
  misc_feature (26)..(26): Cy5 at 3′ end
nnattncnnatnatcnntnnattncn

SEQ ID NO: 57 - DNA, 26 bp, Artificial Sequence
  Synthetic oligonucleotide
  misc_feature (1)..(1): IbRQ at 5′ end
  misc_feature (26)..(26): IbFQ at 3′ end
cgcaatccaccgatcatccgcaatcc

SEQ ID NO: 58 - DNA, 26 bp, Artificial Sequence
  Synthetic oligonucleotide
ggattgcggatgatcggtggattgcg

SEQ ID NO: 59 - DNA, 40 bp, Artificial Sequence
  Synthetic oligonucleotide
cccacacaacaaacaaacaaacaaaacaccaaacaaccac

SEQ ID NO: 60 - DNA, 40 bp, Artificial Sequence
  Synthetic oligonucleotide
gtggttgtttggtgttttgtttgtttgtttgttgtgtggg

SEQ ID NO: 61 - DNA, 30 bp, Artificial Sequence
  Synthetic oligonucleotide
cccacacaacaaacaaacaaacaaaacacc

SEQ ID NO: 62 - DNA, 30 bp, Artificial Sequence
  Synthetic oligonucleotide
ggtgttttgtttgtttgtttgttgtgtggg

(19) United States
(12) Patent Application Publication
(10) Pub. No.: US 2019/0010544 A1
(43) Pub. Date: Jan. 10, 2019

(54) MOLECULAR ZIPPER TWEEZERS AND SPRING DEVICES

(71) Applicant: The Regents of the University of California, Oakland, CA (US)

(72) Inventors: Ratneshwar Lal, La Jolla, CA (US); Preston B. Landon, Rancho Palos Verdes, CA (US); Srinivasan Ramachandran, San Diego, CA (US); Alexander Mo, La Jolla, CA (US)

(21) Appl. No.: 15/952,152

(22) Filed: Apr. 12, 2018

Related U.S. Application Data
(62) Division of application No. 14/003,442, filed on Nov. 18, 2013, now abandoned.
(60) Provisional application No. 61/450,544, filed on Mar. 8, 2011.

(51) Int. Cl. C12Q 1/6876; C12N 15/115; C12Q 1/6818; C12Q 1/6825

(52) U.S. Cl. CPC C12Q 1/6876; C12N 15/115; C12N 2310/16; C12Q 1/6818; C12Q 2537/1373; C12Q 2525/101; C12Q 1/6825

(57) ABSTRACT

Techniques, structures, devices and systems are disclosed for implementing molecular zipper tweezers and springs. In one aspect, a molecular device includes three molecular components including at least a passive side molecular component, a binding side molecular component and a target molecular component adapted to interact together as a zipper that separate two of the molecular components held together by molecular interaction forces.

### CROSS REFERENCE TO RELATED APPLICATIONS

This patent document is a divisional of U.S. patent application Ser. No. 14/003,442 entitled “MOLECULAR ZIPPER TWEEZERS AND SPRING DEVICES” filed on Nov. 18, 2013, which is a 35 USC § 371 National Stage application of International Application No. PCT/US2012/028383 entitled “MOLECULAR ZIPPER TWEEZERS AND SPRING DEVICES” filed Mar. 8, 2012, which claims the priority of U.S. provisional application No. 61/450,544 entitled “MOLECULAR ZIPPER, TWEEZERS AND SPRING DEVICES” filed on Mar. 8, 2011, the entire disclosures of which are incorporated by reference as part of this document.

### STATEMENT REGARDING FEDERALLY SPONSORED RESEARCH OR DEVELOPMENT

This invention was made with government support under grant 5R01DA025296-04 awarded by the National Institute on Drug Abuse (NIDA) of the National Institutes of Health (NIH). The government has certain rights in the invention.

### BACKGROUND

This patent document relates to systems, devices, and processes that use nanoscale molecular sensor and actuator technologies.

Nucleic acids, e.g., deoxyribonucleic acid (DNA) and ribonucleic acid (RNA), can be used to construct various structures for a wide range of applications.

### SUMMARY

Techniques, systems, devices and materials are disclosed for implementing a molecular-based nanoscale sensors and actuators including nucleic acid-based zipper tweezers and springs.

In one aspect of the disclosed technology, a molecular zipper device includes a double-stranded molecule including a first strand of nucleotide units coupled to a second strand of nucleotide units, the nucleotide units of the first strand configured in a sequence and including nucleobases, the nucleotide units of the second strand configured in a complement sequence corresponding to the sequence of the nucleotide units of the first strand, in which at least one nucleotide unit of the second strand includes a synthetic nucleobase that forms a bond with a corresponding complement nucleobase of the first strand, in which the double-stranded molecule is structured to interact with an opening molecule which includes a third strand of nucleotide units in a complementary sequence corresponding to the sequence of the nucleotide units of the first strand, and in which the opening molecule couples to the first strand by unbinding the nucleotide units of the second strand from the nucleotide units of the first strand, the nucleotide units of the third strand having nucleobases that form a substantially equal or stronger bond with the corresponding complement nucleobases on the first strand than the bond formed by the synthetic nucleobase on the second strand.

In another aspect, a molecular sensor device includes a double-stranded molecule including a binding strand and a passive strand, the binding strand including a binding zipper member in connection with a binding hinge member, the passive strand including a passive zipper member in connection with a passive hinge member, in which the passive hinge member is coupled to the binding hinge member, and in which the passive zipper member is coupled to the binding zipper member by a coupling of complementary nucleotide units of the passive zipper member and the binding zipper member, in which the double-stranded molecule is operable to interact with a target molecule initially uncoupled to the double-stranded molecule, the target molecule including an opening strand having nucleotide units in a complement sequence corresponding to a sequence of nucleotide units of the binding zipper member, and in which the opening strand couples to the binding zipper member by uncoupling the complementary nucleotide units of the passive zipper member from the binding zipper member, the nucleotide units of the opening strand bonding to the nucleotide units of the binding zipper member.

Implementations can optionally include one or more of the following features. The molecular sensor device can further include a reset molecule initially uncoupled to the target molecule and the double-stranded molecule, the reset molecule including a closing strand of nucleotide units in a complementary sequence corresponding to the sequence of nucleotide units of the opening strand. The binding strand of the molecular sensor device can further include a binding loop member that connects the binding zipper member to the binding hinge member, and the passive strand of the molecular sensor device can further include a passive loop member that connects the passive zipper member to the passive hinge member, in which the binding loop member and the passive loop member are uncoupled with one another.

In another aspect, a method of capturing a target molecule includes deploying a double-stranded molecule into a fluid environment, the double-stranded molecule including a binding strand having a sequence of nucleotides that is coupled to a passive strand having a complementary sequence of nucleotides, and attaching a target molecule in the fluid environment to the binding strand, the target molecule including an opening strand having a complement sequence of nucleotides corresponding to the binding strand, in which the attaching uncouples the passive strand as the nucleotides of the opening strand bond to the corresponding complement nucleotides of the binding strand.

Implementations can optionally include one or more of the following features. The method can further include removing the target molecule from the double-stranded molecule by coupling the opening strand to a complement closing strand of a reset molecule. The method can further include recoupling the complementary sequence of nucleotides of the passive strand to the sequence of nucleotides of the binding strand, thereby regenerating the double-stranded molecule.

In another aspect, a molecular device includes molecular components including at least a passive side molecular component, a binding side molecular component and a target molecular component, in which the passive side molecular component and the binding side molecular component are bound together by molecular interaction forces to form a molecular zipper structure, in which the target molecular component is initially unbound to the molecular zipper structure and adapted to separate the passive side molecular component and the binding side molecular component.

In another aspect, a molecular actuator device includes a double-stranded molecule including a hinge member attached at one end to a zipper member, the zipper member including a binding strand coupled to a passive strand, in which the binding strand includes a sequence of nucleotide units hybridized a corresponding complement sequence of nucleotide units of the passive strand, a first arm member connected to the binding strand of the zipper member by a first linker strand that attaches the first arm member to the binding strand, and a second arm member connected to the passive strand of the zipper member by a second linker strand that attaches the second arm member to the passive strand.

Implementations can optionally include one or more of the following features. The first arm member can include a double-stranded molecular structure, and the second arm member can include a double-stranded molecular structure. The double-stranded molecule can be structured to interact with a target molecule initially uncoupled to the molecular actuator device, the target molecule including an opening strand having nucleotide units in a complementary sequence corresponding to the sequence of nucleotide units of the binding strand, in which the opening strand couples to the binding strand by uncoupling the complement sequence of nucleotide units of the passive strand from the binding strand and binding the nucleotide units of the opening strand to the nucleotide units of the binding strand. The molecular actuator device can further include a reset molecule initially uncoupled to molecular actuator device, the reset molecule including a closing strand of nucleotide units in a complementary sequence corresponding to the sequence of nucleotide units of the opening strand, in which the closing strand couples to the opening strand by uncoupling the opening strand from the binding strand. The double-stranded molecular structure of the arm member can be structured to interact with another target molecule initially uncoupled to the molecular actuator device, the other target molecule. The molecular actuator device can operate as a spring. The molecular actuator device can be a first molecular actuator device connected to a second molecular actuator device, in which the first arm member and the second arm member of the first molecular actuator device connect with the first arm member and the second arm member of the second molecular actuator device, forming a joined molecular actuator device. The joined molecular actuator device can further include at least one other molecular actuator device, in which the hinge member of the at least one other molecular actuator device connects to a joined arm member of the first and second molecular actuator devices, thereby forming a multiple molecular actuator device. The multiple molecular actuator device can operate as at least one of a motor or a gate element. The molecular actuator device can be incorporated in a capsule, the capsule further including a container unit including a wall that forms an enclosure around an interior region, the container unit structured to include an opening, and a lid unit including a surface structured to cover the opening, in which the molecular actuator device joins the container unit to the lid by a distal end of the first arm member coupled to the surface of the lid and another distal end of the second arm member coupled to an interior surface of the interior region of the container unit. The molecular actuator device of the capsule can include a self-splicing DNA sequence as part of the first arm member that includes a DNAzyme that cleaves a single strand of the double-stranded molecular structure of the first arm member, thereby detaching the lid unit from the capsule. The capsule further can include a material initially enclosed within the capsule, the material released outside the capsule upon detaching the lid unit from the capsule, in which the material can include a drug, imaging agent, enzyme, nucleic acid, viral vector, or other molecular substance.

In another aspect, a DNA based molecular device includes a nanoscale molecular sensor, and a molecular actuator, in which, upon sensing a specific DNA sequence, the nanoscale molecular sensor detects and holds the DNA sequence and the molecular actuator contracts and imparts force to open and close the nanoscale molecular sensor.

Implementations can optionally include one or more of the following features. The nanoscale molecular sensor can operate as tweezers, and the molecular actuator can operate as a spring. The nanoscale molecular sensor and the actuator can be activated under specific environmental conditions including temperature and pH.

The subject matter described in this patent document can be implemented in specific ways that provide one or more of the following features. For example, the disclosed technology can include molecular devices that can sense, hold, and release a target (e.g., DNA) upon specific interaction. For example, the disclosed molecular devices can include exemplary zipper-based tweezers to sense a target (e.g., a DNA strand) and actuate a function. For example, a driving energy to capture an exemplary target DNA strand can be distributed over the entire length of the strand, which can allow more driving energy to be employed, e.g., for holding longer DNA strands and faster opening and closing kinetics. For example, the disclosed zipper-based tweezers can be opened without the use of overhang structures, and thus allow spontaneous regeneration of the exemplary tweezers at its sensing position. For example, the disclosed zipper-based tweezers can be used in the development of new therapeutics and nanoscale machines. For example, the disclosed zipper-based tweezers can include a helix setup to be invaded by natural DNA/RNA for in vitro diagnostics.

### BRIEF DESCRIPTION OF THE DRAWINGS

FIG. 1A (SEQ ID NOS: 59-62) shows schematic illustrations of base pair sequences used in exemplary molecular zippers.

FIGS. 1B-1D show diagrams of the chemical structure of base pair binding in exemplary DNA zippers.

FIG. 2 shows schematics of an exemplary implementation of the disclosed DNA zipper.

FIG. 3 shows a series of schematics demonstrating the structure and function of an exemplary DNA zipper-based tweezers.

FIG. 4A shows a fluorescence spectra plot of exemplary functionalized W strands.

FIG. 4B shows exemplary gel electrophoresis data of the position of dsDNA and ssDNA W strands.

FIG. 5 shows a data plot of time lapse fluorescence spectra of exemplary functionalized W strands at 37° C.

FIGS. 6A-6D show fluorescence spectra plots of exemplary W strands functionalized with the FAM fluorophore on the 5′ end and the Cy5 fluorophore on the 3′ end of the W strand.

FIGS. 7A and 7B show data plots of the time-lapse fluorescence of exemplary functionalized zipper tweezers.

FIGS. 8A-8D show opening and closing cycling data of exemplary zipper tweezers.

FIG. 9 shows a data plot of the normalized fluorescence spectra of exemplary opened zipper tweezers.

FIGS. 10A-10C show comparative data of the closing kinetics of exemplary closing strands.

FIGS. 11A and 11B show schematic illustrations of the disclosed zipper mechanism and zipper based springs technology.

FIGS. 12A and 12B show fluorescent DNA gel electrophoresis data of the transitions exhibited by exemplary zipper springs.

FIGS. 13A-13C show time-lapse fluorescence signal plots and corresponding illustrative schematics for exemplary zipper springs.

FIGS. 14A and 14B show time-lapse fluorescence spectra plots from successive extension and contraction cycles of exemplary zipper springs.

FIGS. 15A and 15B show time-lapse fluorescence signal plots of the extension of exemplary zipper springs with inosine-containing extending strands and in a zipper-less spring configuration.

FIG. 16 shows a time-lapse fluorescence plot demonstrating the contraction function of exemplary zipper springs.

FIGS. 17A and 17B show illustrative schematics and time-lapse fluorescence measurement plots of exemplary zipper springs activity upon releasing the arm member.

FIG. 18 shows DNA gel determination data and corresponding illustrations of arm member removal from exemplary zipper springs in contracted to extended states.

FIG. 19 shows DNA gel determination data and corresponding illustrations of exemplary zipper springs after arm member removal.

FIG. 20A shows an exemplary double zipper structure.

FIG. 20B shows an exemplary zipper array structure.

FIG. 21 shows an exemplary DNA zipper position motor.

FIG. 22 shows an exemplary channel gating DNA zipper structure.

FIGS. 23A-23C shows schematic illustrations of exemplary controlled drug delivery devices employing the disclosed zipper mechanism.

Like reference symbols and designations in the various drawings indicate like elements.

### DETAILED DESCRIPTION

Techniques, systems, devices and materials are disclosed for implementing molecular-based nanoscale sensors and actuators including nucleic acid-based zipper tweezers and springs.

Nucleic acids, e.g., deoxyribonucleic acid (DNA) and ribonucleic acid (RNA), can be used to create a variety of molecular machines, with properties mimicking logic-circuit operations. For example, the small size, high binding specificity, ease of chemical synthesis and availability of inexpensive DNA or RNA oligonucleotides can make DNA/RNA-based molecular devices useful in a variety of applications. For example, the specificity with which DNA hybridizes can be applied for designing a variety of DNA based diagnostic and therapeutic systems.

A naturally-occurring double-stranded DNA (dsDNA) includes a linked chain of deoxyribose sugar as a backbone for four nucleotide bases (also referred to as nucleobases), e.g., including adenine (A), cytosine (C), guanine (G), thymine (T). These four nitrogen bases can form hydrogen bonds that hold two individual strands of the DNA together. For example, in naturally-occurring dsDNA, adenine bonds to thymine (A=T) and cytosine bonds to guanine (C≡G). The A=T and C≡G bonds are two different types of hydrogen bonds formed by the base pairs. Adenine forms two hydrogen bonds with thymine (A=T) and cytosine forms three hydrogen bonds with guanine (C≡G). For example, the energy of formation of N—H . . . O bonds is approximately 8 kJ/mol, and the energy of formation of N—H . . . N bonds is approximately 13 kJ/mol (e.g., where the dotted line represents the hydrogen bond). A naturally-occurring RNA molecule includes a linked chain of ribose sugar as a base for four nucleobases, e.g., including A, C, G, and uracil (U). For example, when RNA binds to DNA, an adenine nucleobase of DNA forms two hydrogen bonds with uracil nucleobase of RNA (A=U). RNA molecules are single stranded and can form many structural configurations.

The disclosed technology can include molecular tweezers and molecular springs to sense a target and actuate a function. For example, the disclosed molecular tweezers and molecular springs can be based on nucleotide zipper mechanisms where molecular bonds can be engaged or disengaged/released as zippers. For example, an exemplary zipper can be used to create a DNA nano-gate that can be reversibly opened and closed. The disclosed molecular zipper technology can include self-sustaining, modifiable properties that can be implemented in sensing and actuating applications exhibiting sensitivity in a range of physiologically relevant temperatures. For example, the disclosed molecular zipper technology can be implemented in various nanoscale applications, e.g., including molecular motor actuation, molecular recognition tools (e.g., molecular detection assays and molecular and biological sensors, molecular building blocks, vehicles for molecular transport (e.g., colloidal drug carriers) and as molecules modifiers and medicines.

In one aspect, the disclosed technology can include devices, systems, and techniques based on nucleotide zipper mechanisms. For example, an exemplary molecular zipper can include a closed double helix molecule (e.g., DNA) formed by the hybridization of two strands of oligonucleotides that can be opened by the capture of a target molecule, e.g., such that the double-strand separation does not use external energy. For example, the exemplary double helix molecule can include a binding strand having naturally-occurring nucleotides and a passive strand including non-naturally-occurring nucleotides. For example, the molecular zipper mechanism can be implemented by the target molecule (e.g., also referred to as an opening strand, an external strand, and a fuel strand) hybridizing with the binding strand, e.g., displacing the passive strand. For example, the passive strand does not bond to the binding side of the exemplary molecular zipper as strongly as the target molecule. The disclosed technology can function like a ‘zipper’ because the closed double helix molecule can naturally separate by interacting with the target. The physical interactions that take place between the target molecule and a closed molecular zipper can open the exemplary molecular zipper.

As a specific example, an exemplary DNA double helix can include one oligonucleotide strand that can be referred to as the normal strand (N) and the other oligonucleotide strand that can be referred to as the weak strand (W). In some implementations, the exemplary N strand can be a natural DNA strand, e.g., including the four naturally-occurring DNA nucleobases: adenine (A), cytosine (C), guanine (G), and thymine (T). For example, the exemplary N strand can be a natural RNA strand, e.g., including the four naturally-occurring RNA nucleobases: A, C, G, and uracil (U). The exemplary W strand can be an engineered or synthetic strand having a sequence of bases that includes non-naturally-occurring nucleobases. For example, the non-naturally-occurring nucleobases on the exemplary W strand can be configured to provide a weaker binding affinity to their corresponding complement nucleobases compared to the binding affinity between two naturally-occurring nucleobases. For example, when the exemplary N and W strands hybridize, there is less energy holding N and W strands together than if the W strand comprised the corresponding natural complement nucleobases of the N strand. For example, the exemplary W strand (also referred to as a synthetic strand, an engineered strand, and a passive strand) can be constructed using a deoxyribose sugar backbone identical to that occurring in natural DNA, but containing only nucleotide analog bases—nucleotide analogs are bases that can be attached to the backbone (e.g., the deoxyribose sugar backbone), but do not naturally occur in organisms.

For example, an exemplary opening strand (O) can be the natural complement of the exemplary N strand and thereby displace the W strand at each nucleotide unit along the W strand. In some examples, the exemplary O strand can include the same number or a greater number of nucleotide units than the exemplary W strand, e.g., in which the O strand hybridization with the N strand can detach the W strand from the double helix molecule. In other examples, the exemplary O strand can include a smaller number of nucleotide units than the exemplary W strand, e.g., in which the W strand can remain attached to the exemplary N strand (and part of the double helix molecule) after the O strand hybridization with the N strand.

The disclosed technology can include a variety of W strands that can be configured to provide differing binding affinities of the W strand to the N strand. In some examples, the exemplary W strand can be configured to have all of its nucleotide bases to be non-naturally-occurring nucleobases. In other examples, the exemplary W strand can be configured to have some of its nucleotide bases to be non-naturally-occurring nucleobases, e.g., spatially organized in a desired sequence with naturally-occurring nucleobases. For example, non-naturally-occurring nucleobases can include inosine (I), 2-aminopyrimidine, 5-methyisocytosine, and deoxyinosine, among others. For example, an exemplary W strand can contain the inosine (I) base along with other naturally-occurring bases. The exemplary W strands can be engineered to have differing affinities to any N strand, e.g., providing flexibility in the disclosed zipper-based devices that can also self regenerate.

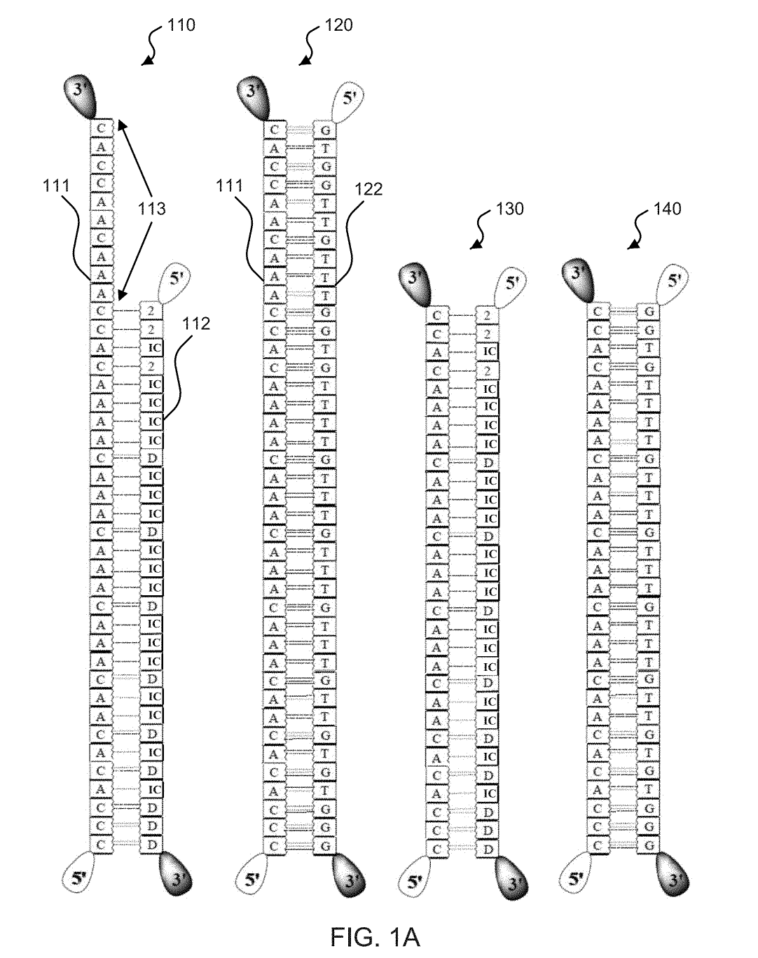

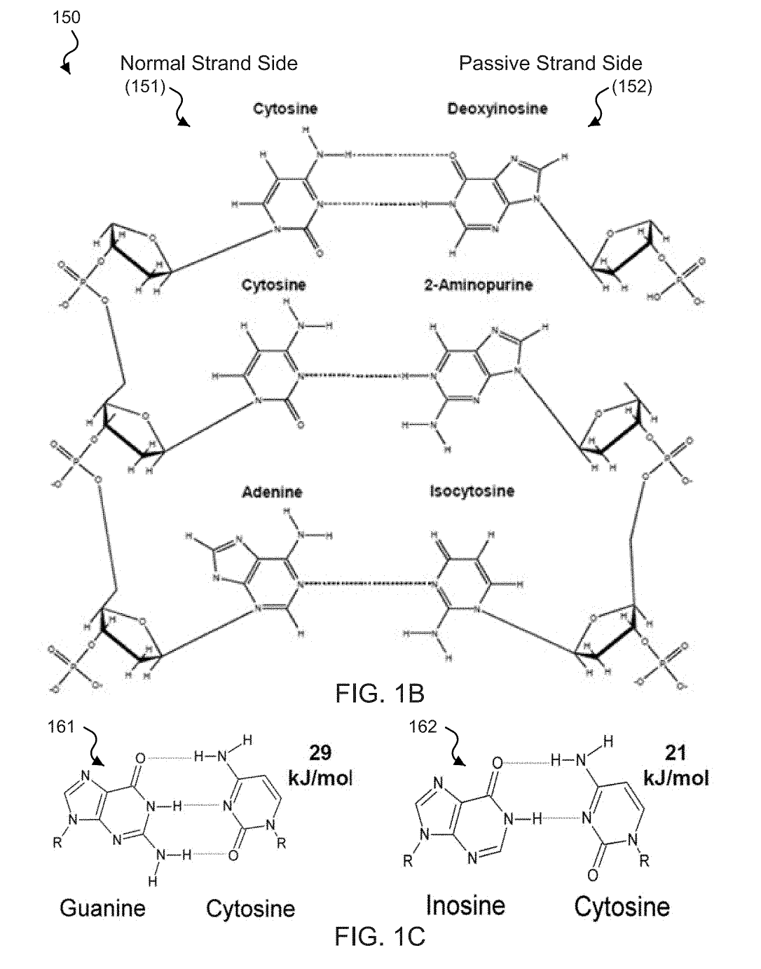

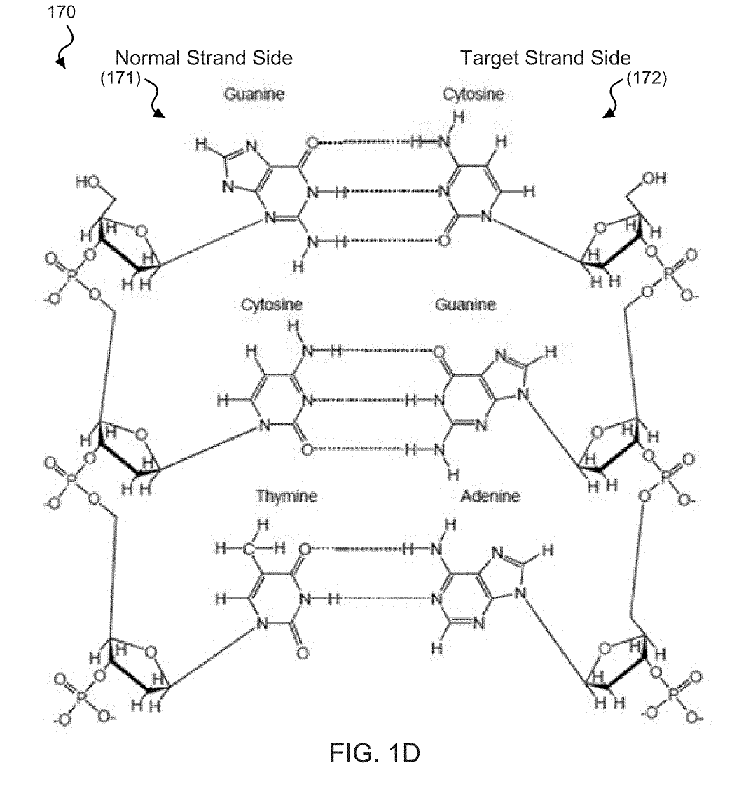

FIG. 1A shows diagrams of exemplary double-stranded helices 110, 120, 130, and 140 including base pair sequences that can be used to create an exemplary molecular zipper-based devices. For example, the exemplary double-stranded helices 110, 120, 130, and 140 can represent dsDNA, RNA hybridized to another oligonucleotide strand, or other configuration. The double-stranded helix 110 shows a binding strand 111 including naturally-occurring DNA nucleobases hybridized to a weak strand 112 (e.g., also referred to as a passive strand) that include non-naturally-occurring nucleobases, e.g., featuring 2-aminopyrimidine (2), 5-methyisocytosine (IC), and deoxyinosine (D). The exemplary dotted lines connecting the bases between the two strands represent hydrogen bonds that can form between the two complementary nucleobases and hybridize the different strands. In this example, the binding strand 111 includes an extra sequence of nucleotide units referred to as a tab (e.g., tab 113, shown between the arrows at the top of the binding side of the zipper). The double-stranded helix 120 shows the binding strand 111 hybridized to a complementary strand 122, e.g., which can be an opening strand used to unzip a passive strand (e.g., the weak strand 112) from the binding strand 111. The exemplary diagram featuring the double-stranded helix 120 shows an increased number of hydrogen bonds between the strands in the dsDNA 120 and than in the dsDNA 110. For example, the double-stranded helix 110 can represent a dsDNA in which the left strand of the helix (e.g., the binding strand 111) depicts the sequence of the binding side of the zipper while the right strand of the helix (e.g., the weak strand 112) depicts the passive side of the zipper. For example, the tab 113 can be used to match a sequence on a target molecule that can start the unzipping process. The exemplary diagram featuring the double-stranded helix 120 shows the binding strand 111 remains unchanged after zipping the complementary strand 122 and depicts the nucleotide units of the tab 113 hybridized to their corresponding complement nucleotide units of the complementary strand 122, in which the tab 113 assisted in facilitating the zipper mechanism after the passive side has been displaced and replaced by the stronger binding target strand. The exemplary diagrams featuring the double-stranded helices 130 and 140 are similar to the exemplary diagrams of the double-stranded helices 110 and 120, except the bonding between the binding side of the zipper is not facilitated with an unpaired tab sequence at a region of the zipper.

FIG. 1B shows an exemplary diagram 150 of the chemical structure of base pair binding between naturally-occurring and non-naturally-occurring bases, which can be implemented in an exemplary DNA zipper based on the disclosed technology. For example, the diagram 150 features a normal strand side 151 including a sequence of naturally-occurring DNA nucleobases C-C-A coupled to a passive strand side 152 including a complementary sequence of non-naturally-occurring DNA nucleobases D-2-IC. The exemplary dotted lines connecting the bases between the two strands represent hydrogen bonds formed between the complementary nucleobases. For example, two hydrogen bonds can form between C=D nucleobases, and only one hydrogen bond can form between C-2 and A-IC nucleobases.

Exemplary DNA based zippers can also be configured using inosine. For example, inosine preferentially hybridizes to C through two hydrogen bonds. The exemplary I═C pair has a weaker energy of formation (˜21 kJ/mol) than the G≡C pair (˜29 kJ/mol). Exemplary W strand can be configured to contain the inosine base along with other naturally-occurring bases. For example, when an exemplary N strand and the inosine-containing complementary W strand hybridize, there is less energy holding them together, e.g., than if they were the exemplary N strand and its natural complement. For example, the stronger G≡C interaction between an exemplary natural complement and the exemplary N strand outcompetes the I═C bonds and displaces the exemplary W strand from the exemplary DNA zipper structure, e.g., resulting in the opening of the zipper, to form a new double stranded DNA structure having the N strand coupled to its natural complement strand.

FIG. 1C shows exemplary diagrams 161 and 162 of the chemical structure of base pair binding, e.g., which can be implemented in an exemplary DNA zipper of the disclosed technology. The exemplary diagram 161 shows the bonding structure between the naturally-occurring nucleobases guanine and cytosine. For example, the bonding energy between C≡G is 29 kJ/mol. The exemplary diagram 162 shows the bonding structure between the naturally-occurring nucleobase cytosine and the non-naturally-occurring nucleobase inosine (I). For example, the bonding energy between C═I is 21 kJ/mol, which is substantially less than the bonding energy of the C≡G boding pair.

FIG. 1D shows an exemplary diagram 170 of the chemical structure of base pair binding between naturally-occurring bases, e.g., which can be implemented in an exemplary DNA zipper of the disclosed technology. For example, the diagram 170 features a normal strand side 171 including a sequence of naturally-occurring DNA nucleobases G-C-T coupled to a target strand side 172 including a complementary sequence of naturally-occurring DNA nucleobases C-G-A. The exemplary dotted lines connecting the bases between the two strands represent hydrogen bonds formed between the complementary nucleobases. For example, two hydrogen bonds can form between T=A nucleobases, and three hydrogen bonds can form between C≡G nucleobases. For example, for this reason, the nucleotide units in the weak strand 112 of the zipper in FIG. 1A cannot generate as much bonding energy between the binding strand 111 as the complementary strand 122 can with the binding strand 111.

The described molecular zippers can be composed of three molecular components that include a passive side, a binding side and a target that are entropy driven to interact in such a way that they perform the function of separating two individual parts held together by molecular interaction forces. For example, interaction forces can include any combination of hydrogen bonds, van der Waals attraction, hydrophobic interactions or electrostatic forces existing between the interacting molecular components. The passive and binding sides can be initially bound together forming a zipped molecule. The passive side of the molecular zipper can be separated from the binding side by interaction with the target (e.g., displaced at each nucleotide unit that the target binds to the binding side) through a process called entropy driven displacement (EDD). This exemplary separation of the passive side from the binding side is a function of the exemplary molecular zipper device. For example, the exemplary molecular zipper device can be described as being opened by a molecular key that does not require the addition of any energy. For example, the exemplary molecular zipper can be opened by a chemically engineered molecular key, or the exemplary molecular zipper can be chemically engineered to be opened by a naturally-occurring molecule to act as the key.

For example, physical principles involved in the opening of the molecular zipper include thermal fluctuations between the two individual strands of the zipper and molecular forces between the components of the zipper. The disclosed molecular zipper mechanism can rely on thermal fluctuations between the base pairs as well as the bonding energies between the three components. For example, the molecular zipper can be opened by allowing the target to statistically wiggle its way into the zipper by pushing the passive side out of the zipper. For the molecular zipper mechanism to function, the average energy of interaction between the binding side of the zipper and the target is greater than the average energy of interaction between the binding side and the passive side. In addition, the increased attraction between the binding side and the target can occur with a periodicity close enough together so that the thermal fluctuations that facilitate the opening action are statistically probable. For example, provided that the periodicity of increased bonding between the target and the binding side of the zipper occurs within statistical reason and the bonding energy between the passive side and the target are negligible, the driving energy of the unzipping action can be approximated. For example, the approximate total driving energy of the unzipping action (Eᵤ) can be represented by Eq. (1):

Eᵤ=Eₜ-Eₚ (1)

where Eₜ is the total bonding energy between the target and the binding side and Eₚ is the total bonding energy between the passive side and the binding side. The total driving energy of the unzipping action, e.g., represented in Eq. (2), can become:

Eᵤ=[Mₜ(8 kJ/mol)+Nₜ(13 kJ/mol)]−[Mₚ(8 kJ/mol)+Nₚ(13 kJ/mol)] (2)

where M and N represent the number of hydrogen bonds of the form N—H . . . O and N—H . . . N, respectively.

For example, the average thermal kinetic energy of a molecule is given by E=nRT where n is the number of moles, R is 8.3145 and T is the temperature in Kelvin (K). Physiological temperature is approximately 300 K, and the minimum average molecular kinetic energy at this temperature is E=2.5 kJ/mol. For example, since the biding energy of the hydrogen bonds is only several times larger than their disassociation tendency due to thermal motion, the hydrogen bonds between the nucleosides in dsDNA are constantly breaking and reforming. For example, this causes the DNA to temporarily undergo localized distortions and deformations. For example, intercalating agents such as ethidium bromide can insert into dsDNA with ease, which can suggests that the double-stranded helix temporally unwinds and presents gaps for these agents to occupy. Thus, the DNA conformation can be represented by a flickering repertoire of dynamic structures. For example, this can suggest that the ends of the two strands in a double helix must continuously undergo breaking, partially unwinding and reforming due to thermal fluctuations. For example, since the bond energy between one hydrogen bond (e.g., ˜10 kJ/mol) is only approximately 5 times grater then the thermal fluctuation energy at physiological temperatures (e.g., ˜2.5 kJ/mol), a single hydrogen bond in a double-stranded helix can be expected to be bonding only ⅘ of the time and thus be temporarily broken ⅕ of the time. It then follows, for example, that for any time sufficient in length, the probability P of n consecutive hydrogen bonds being simultaneously broken at the front of the front of a dsDNA helix is P=(⅕)ⁿ.

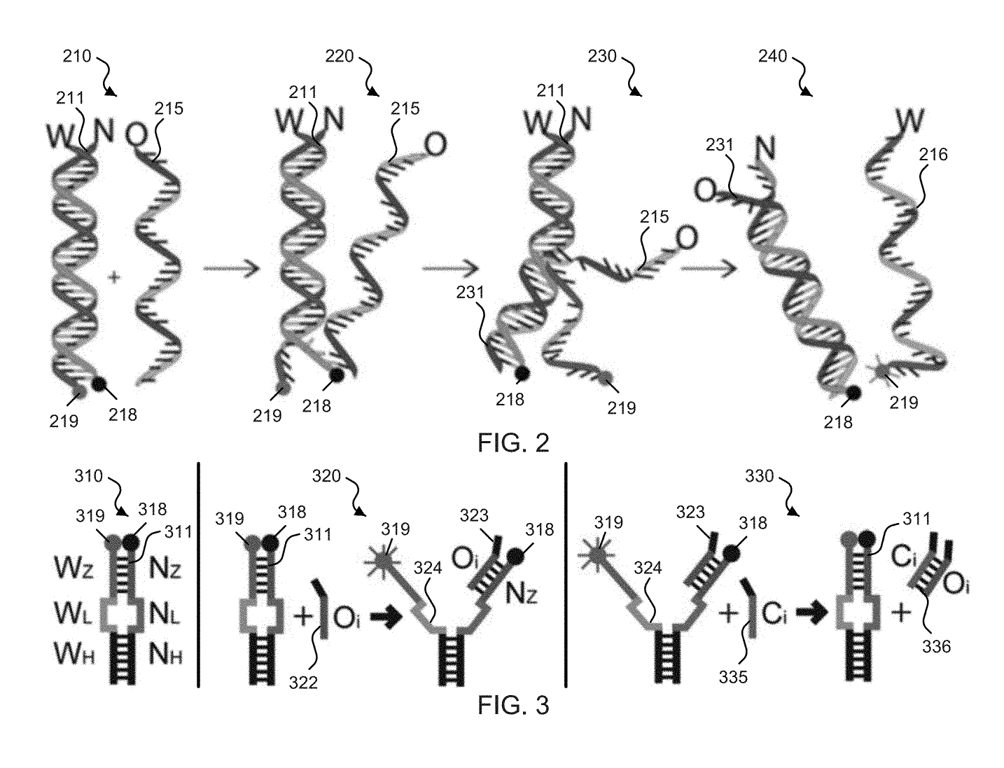

FIG. 2 shows a series of schematics of an exemplary implementation of the exemplary zipper mechanism in the disclosed DNA zipper tweezers device. For example, a schematic 210 shows a double-stranded zipper [N:W] helix 211 (e.g., with a normal single strand of DNA (N strand) coupled to a passive synthetic nucleotide strand (W strand) 216) that is weakly bound together, e.g., due to fewer hydrogen bonds between the I═C base pairs. The schematic 210 also shows an opening strand (O strand) 215 that is the natural complement of the N strand. A schematic 220 shows the introduction of the O strand 215 to the double-stranded zipper helix 211. A schematic 230 shows the double-stranded zipper [N:W] helix 211 being invaded by the O strand 215 and the formation of a double-stranded zipper [N:O] helix 231 that includes a higher binding energy between bases than the double-stranded zipper [N:W] helix 211. For example, when the W strand 216 and the exemplary N strand hybridize, there is less energy holding them together in the double-stranded zipper [N:W] helix 211 than the exemplary N strand and the O strand 215 in the double-stranded zipper [N:O] helix 231. For example, upon introduction of the O strand 215 to [N:W] helix 211, the stronger G≡C interaction out competes the I═C bonds and the O strand 215 replaces the exemplary W strand 216 in the helix resulting in ‘opening of the zipper’. A schematic 240 shows the more stable double-stranded zipper [N:O] helix 231 formed and the separation of the W strand 216. This exemplary interaction can be summarized in Eq. (3):

[N:W]+O→[N:O]+W (3)

An exemplary comparison of the hydrogen bond energies of [N:W] and [N:O] suggests approximately 140 kJ/mol is driving the reaction of Eq. (3), e.g., assuming ˜21 kJ/mol for the I═C bond and ˜29 kJ/mol for the C-G bond. For example, the W strand 216 can be configured such that to distribute of the energy along the length of the strand, e.g., periodic spacing of I with a sufficient spatial frequency along the length of the W strand can be configured for the operation of the zippers. For example, the thermal stability, kinetics and specificity of the zipper are dependent on the number of I═C bonds, their order and period of placement.

Also shown in FIG. 2, exemplary fluorophores 218 and 219 can be bound to the individual strands. For example, the exemplary fluorophore 218 is attached to the N strand can be a quencher that quenches the exemplary fluorophore 219 attached to the W strand when the double-stranded zipper helix 211 is in a zipped position. For example, the exemplary fluorophore 219 can fluoresce when the N strand and the W strand become uncoupled, e.g., indicating that the double-stranded zipper helix is unzipped.

Table 1 shows exemplary DNA oligonucleotides base pair sequences for the individual strands of the zipper system. For example, bases presented in lower case represent the sight of a base pair mismatch in the opening strand.

TABLE 1

| | SEQ | |
|---|---|---|
| | ID | |
| Name | NO | Sequence |
| W | 1 | 5′-FAM/IIT ITT ITT TIT TIT TII TTT |
| | | IIT TTI TTI TIT TTI II/Cy5-3′ |
| N | 2 | 5′-/IBRQ/CCC AAC CAC AAC AAA CCA AAC |
| | | CAA CAA CAA ACA ACA CC/IBFQ/-3′ |
| O | 3 | 5′-GGT GTT GTT TGT TGT TGG TTT GGT |
| | | TTG TTG TGG TTG GG-3′ |
| O_{M1} | 4 | 5′-GaT GTT aTT TGT TaT TGG TTT aGT |
| | | TTG TTa TGG TTa GG-3′ |
| O_{M2} | 5 | 5′-aaT aTT GTT TaT TGT TaG TTT GaT |
| | | TTG TTa TGa TTG aG-3′ |
| O_{M3} | 6 | 5′-GaT GTT aTT TGT TaT TGa TTT aGT |
| | | TTa TTG TGa TTG aG-3′ |
| O_{M4} | 7 | 5′-GtT GTT tTT TGT TGT TGt TTT tGT |
| | | TTt TTG TtG TTG tG-3′ |
| O_{M5} | 8 | 5′-ttT GTT tTT TGT TtT TGG TTT tGT |
| | | TTG TTt TGt TTG tt-3′ |
| O_{M6} | 9 | 5′-TTG TGG TGG GTG GTG GTT GGG TTG |
| | | GGT GGT GTT GGT TT-3′ |

In another aspect, the disclosed technology can include devices, systems, and techniques that can provide a DNA based nanoscale sensor, e.g., DNA zipper tweezers. For example, upon sensing a specific DNA sequence (e.g., a target molecule), the exemplary DNA zipper tweezers can detect and hold the target and subsequently release the target, e.g., returning to the initial position. FIG. 3 shows a series of schematics of the structure and function of an exemplary DNA zipper-based tweezers, e.g., implemented to detect, capture, hold, and release a target.

For example, as shown in FIG. 3, a schematic 310 shows a closed DNA zipper-based tweezers 311, e.g., in a zipped or closed position. The closed DNA zipper-based tweezers 311 can be configured using a normal strand (N_{T}) and a weak strand (W_{T}), e.g., each including three members. For example, the N_{T} can include a normal strand zipper arm member (N_{Z}), a normal strand loop member (N_{L}), and a normal strand hinge member (N_{H}). The W_{T} can include a weak strand zipper arm member (W_{Z}), a weak strand loop member (W_{L}), and a weak strand hinge member (W_{H}). In some examples, the N_{T} and W_{T} can be configured with 54 nucleotide units (nt). For example, the exemplary zipper arm members N_{Z} and W_{Z} can contain a 21 nt zipper section; the exemplary hinge members N_{H} and W_{H} can contain a 21 nt hinge section; and the exemplary loop members N_{L} and W_{L} can contain a 12 nt loop section, e.g., intervening the zipper members and hinge members. The exemplary closed DNA zipper-based tweezers 311 can be functionalized at the zipper end, e.g., with a fluorophore 319 (e.g., a Cy5.5 or other fluorophore) attached to W_{Z} and a quencher 318 (Iowa Black RQ (IBRQ)) attached to N_{Z}. For example, the fluorophores are quenched when the exemplary zipper tweezers are in the closed position (e.g., as shown in schematic 310).

For example, as shown in FIG. 3, a schematic 320 shows the closed DNA zipper-based tweezers 311 and a single-stranded opening strand Oᵢ 322 coming together on the left side of the arrow. For example, on the right side of the arrow, the opening strand Oᵢ 322 is shown to open (e.g., unzip) the DNA zipper-based tweezers 311 using the described zipper mechanism, e.g., resulting in an unzipped DNA zipper-based tweezers 324 that can hold/capture a target. For example, the zipper arm members N_{Z} and W_{Z} are hybridized in the closed position (e.g., as shown in the schematic 310 and left side of the arrow in the schematic 320), but are uncoupled after implementation of the disclosed zipper mechanism. For example, the loop members N_{L} and W_{L} can be configured to never hybridize together, e.g., by producing the loop members N_{L} and W_{L} to be non-complementary. For example, the exemplary N_{H} and W_{H} can be configured to remain hybridized during implementations of the exemplary DNA zipper-based tweezers, e.g., by producing the hinge members N_{H} and W_{H} to be tightly bound natural complements. For example, the unzipped DNA zipper-based tweezers 324 can include the generation of a fluorescent signal by the uncoupled fluorophore 319. Also, for example, the opening strand Oᵢ 322 can contain a 7 nt overhang (e.g., overhang nucleotides 323), e.g., to facilitate the opening strand Oᵢ 322 removal.

For example, as shown in FIG. 3, a schematic 330 shows a closing strand Cᵢ 335 and the unzipped DNA zipper-based tweezers 324 coming together on the left side of the arrow. For example, on the right side of the arrow, the closing strand Cᵢ 335 is shown hybridized with the opening strand Oᵢ 322 previously coupled to the unzipped DNA zipper-based tweezers 324, e.g., forming a product double stranded (Oᵢ:Cᵢ) 336 and resetting the unzipped DNA zipper-based tweezers 324 to its zipped or closed position as closed DNA zipper-based tweezers 311. For example, the opening strand Oᵢ 322 competitively displaces the zipper arm member W_{Z}, and the closing is facilitated by removal of the opening strand Oᵢ 322 by the closing strand Cᵢ 335. FIG. 3, by way of example, demonstrates the opening of the disclosed molecular zipper tweezers, e.g., activated by the introduction of an opening strand (e.g., the opening strand Oᵢ 322, shown in the schematic 320), and the closing of the disclosed molecular zipper tweezers, e.g., activated by a closing strand (e.g., the closing strand Cᵢ 335, shown in the schematic 320).

Exemplary implementations were performed to demonstrate the described functionalities and capabilities of the disclosed molecular zipper tweezers. Chemicals used in exemplary implementations were obtained from Sigma Aldrich (Saint Louis, Mo.) unless otherwise specified. The exemplary DNA constructs were obtained from IDT (Coreville, Iowa); the exemplary DNA ladders were obtained from Promega (Madison, Wis.); and the exemplary DNA gels were obtained from Lonza (Walkersville, Md.).

Table 2 shows base pair sequences of the individual component of the exemplary zipper tweezers system, e.g., used in exemplary implementations of the disclosed technology. The exemplary ‘+’ symbol in front of a base in Table 2 indicates that base is a locked nucleic acid (LNA). Text in parentheses represents an exemplary ssDNA overhang.

TABLE 2

| | SEQ | Nucle- | |
|---|---|---|---|
| | ID | otide | |
| Name | NO | Units | Sequence |
| W_{T} | 10 | 54 nt | 5′-Cy5.5/TII ITT IIT ITT ITT |
| | | | TII TTT CTT CTT TCT TCT TGA |
| | | | CCA GTC GCA TGG ATC GGC-3′ |
| N_{T} | 11 | 54 nt | 5′-GCC GAT CCA TGC GAC TGG |
| | | | TCA TTT CCC TCT CCC AAA CCA |
| | | | AAC AAC ACC AAC CCA/IBRQ/-3′ |
| O₁ | 12 | 28 nt | 5′-(AGG AGA A)TG GGT TGG TGT |
| | | | TGT TTG GTT T-3′ |
| C₁-LNA | 13 | 21 nt | 5′-ACA ACA C+CA A+CC +CA+(T |
| | | | T+CT C+CT)-3′ |
| C₁-DNA | 14 | 21 nt | 5′-ACA ACA CCA ACC CA(T TCT |
| | | | CCT)-3′ |
| O₂ | 15 | 32 nt | 5′-GT GTT GTT TGG TTT GGG |
| | | | AGA GGG (TCT CCT TTC)-3′ |
| C₂ | 16 | 32 nt | 5′-(GAA AGG AGA) CCC TCT |
| | | | CCC AAA CCA AAC AAC AC-3′ |
| O₃ | 17 | 24 nt | 5′-GT GTT GTT TGG TTT GGG |
| | | | AGA GGG A-3′ |
| O₃-FAM | 18 | 24 nt | 5′-FAM/GT GTT GTT TGG TTT |
| | | | GGG AGA GGG A-3′ |
| C₃-LNA | 19 | 24 nt | 5′-T+CC +CT+C T+CC +CA+A |
| | | | A+CC AAA CAA CAC-3′ |
| C₃-DNA | 20 | 24 nt | 5′-TCC CTC TCC CAA ACC AAA |
| | | | CAA CAC-3′ |
| C₄-LNA | 21 | 24 nt | 5′-TCC +CT+C TC+C CA+A A+CC |
| | | | A+AA +CAA +CAC-3′ |
| O_{c} | 22 | 21 nt | 5′-TGG GTT GGT GTT GTT TGG |
| | | | TTT-3′ |
| C_{c} | 23 | 21 nt | 5′-AAA CCA AAC AAC ACC AAC |
| | | | CCA-3′ |

Exemplary measurements of the melting temperature (Tₘ) were performed in the exemplary implementations. For example, the Tₘ of an initial zipper helix (e.g., [N:W]) and the final state helix (e.g., [N:O]) were measured to be 54° C. and 71° C., respectively, e.g., using an AVIV 202 Circular dichroism spectrometer with a Peltier temperature controller and pH meter. Exemplary measurements were conducted using a double helix concentration of 20 μM suspended in a 10 mM PBS buffer (e.g., pH 7.4, 160 mM NaCl). Exemplary Tₘ calculations of natural DNA pairs were performed using the IDT online calculator with 160 mM NaCl, e.g., assuming equal concentration of 0.1 μM for both strands. Exemplary DNA calculations of sequences containing deoxyinosine were performed using deoxyadenine in the place of deoxyinosine to obtain approximate values for zipper construction. Calculated values were found to be with in a few degrees of our measured values.

Exemplary measurements of the zipper mechanism activity were performed in the following manner. For example, zipper action was visualized by tagging N and W strands with fluorescent probes and observing the change in fluorescence with time. For example, fluorescent quenchers were placed at both ends of the N strands (e.g., 3′-IBFQ and 5′-IBRQ); and 6-carboxyfluorescein (FAM) and Cy5 were placed on W strands at 5′ and 3′ ends, respectively; while O was left unlabeled, e.g., as shown in Table 1. Exemplary fluorescence measurements were conducted using a Jobin Yvon FluoroMax-3 luminescence spectrometer. For example, fluorescent observations (Excitation/Emission) of FAM were performed at 495/520 nm, of Cy5 were performed at 648/688 nm, and of Cy5.5 were performed at 668/706 nm. Exemplary measurements were performed using quartz cuvettes with 40 μL sampling volume (e.g., Sterna Cell 16.40F-Q-10/Z15) filled with 100 μL of sample at the start of each experiment. Exemplary experimental implementations were carried out on samples dissolved in nuclease free reaction buffer (e.g., 30 mM Tris-HCl, 160 mM NaCl, and pH 8.0). Basal fluorescence of the quenched zipper was measured on each sample prior to data collection. For example, basal fluorescence in the exemplary implementations is a measure of the degree of colocalization of the quencher and Cy5.5, e.g., in a closed zipper tweezers. Basal fluorescence can represent the minimum fluorescence of the system prior to any dilution effects. The data was collected typically at every second for ˜90 min and at every 5 s for experiments involving more than 90 min. Exemplary zipper-opening implementations were conducted by adding 10 times more opening strands than zippers, unless stated otherwise. Exemplary initial tweezers-opening implementations were performed by adding 10 times more opening strand, and successive opening and closing experiments were performed by consecutively adding 2 times more of each strand, unless stated otherwise (as shown in Tables 3 and 4). For example, after the initial opening of the zipper tweezers, successive opening and closing cycles were conducted by adding 30 and 50 times Oᵢ opening constructs and 20 and 40 times Cᵢ closing strands, respectively. For example, excessive concentrations can ensure that the reactions can be stabilized with a terminating value and drive the reactions to completion significantly faster than equal concentrations.

TABLE 3

| | Time Taken to Complete 50% |
|---|---|
| Opening Constructs | of the Opening Reaction (t_{1/2}) with |
| (concentration) | Different Loop Binding (L) or Toe (T) Lengths |
| Zipper O (10x) | 195 s |
| O₁ (10x) | 119 s/7 T |
| O₂ (10x) | 26 s/9 L/9T |
| O₃ (10x) | 10 s/10 L |
| O₃ (1x) | 15 s/10 L |

Table 3 shows the kinetics of the opening reaction with different constructs at 37° C.

TABLE 4

| | | Time Taken to Complete |
|---|---|---|
| | Tweezers | 50% of the Closing |
| Closing Strand | Opening Constructs | Reaction at 37° C. |
| (concentration) | (concentration) | with Different Toe (T) lengths |
| C_{1-LNA} (20x) | | 10 s/7 T |
| C_{1-DNA} (20x) | O₁ (10x) | 320 s/7 T |
| C₂ (20x) | O₂ (10x) | 32 s/9 T |
| C_{3-LNA} (10x) | O₃ (2x) | 1.2 h/n |
| C_{4-LNA} (10x) | O₃ (2x) | 6.7 h/n |

Exemplary gel electrophoresis analyses of the exemplary DNA zipper tweezers were performed in the following manner. For example, the initial and final states of the zipper system were confirmed by DNA gel electrophoresis. For example, the final double helix conformation [N:O] was created by thermally annealing [N:W]+10 O the oligonucleotides (e.g., to ensure the reaction was driven to completion) and used as a control sample. Thermal annealing was accomplished using a custom program in a PCR thermocycler (e.g., Mastercycler personal, Eppendorf) to quickly raise the solution temperature to 94° C. beyond the double strand melting temperature (e.g., N:W 54° C.; N:O 71° C.), followed by a slow, controlled, cooling at a rate of 1° C./2 min to a final temperature of 4° C. DNA gel electrophoresis was performed with 4% agarose gel at 5 V/cm in 1× Tris/Borate/EDTA (TBE) buffer while monitoring the solution temperature not to exceed 20° C. For example, in order to resolve single and double-stranded DNA, the positions of the strands within the gel were determined using fluorescent gel imaging and Ethidium Bromide (EtBr) staining. Exemplary gels were imaged with a Bio-Rad FX-Imager Pro Plus and analyzed with the Quantity One software package (Bio-Rad).

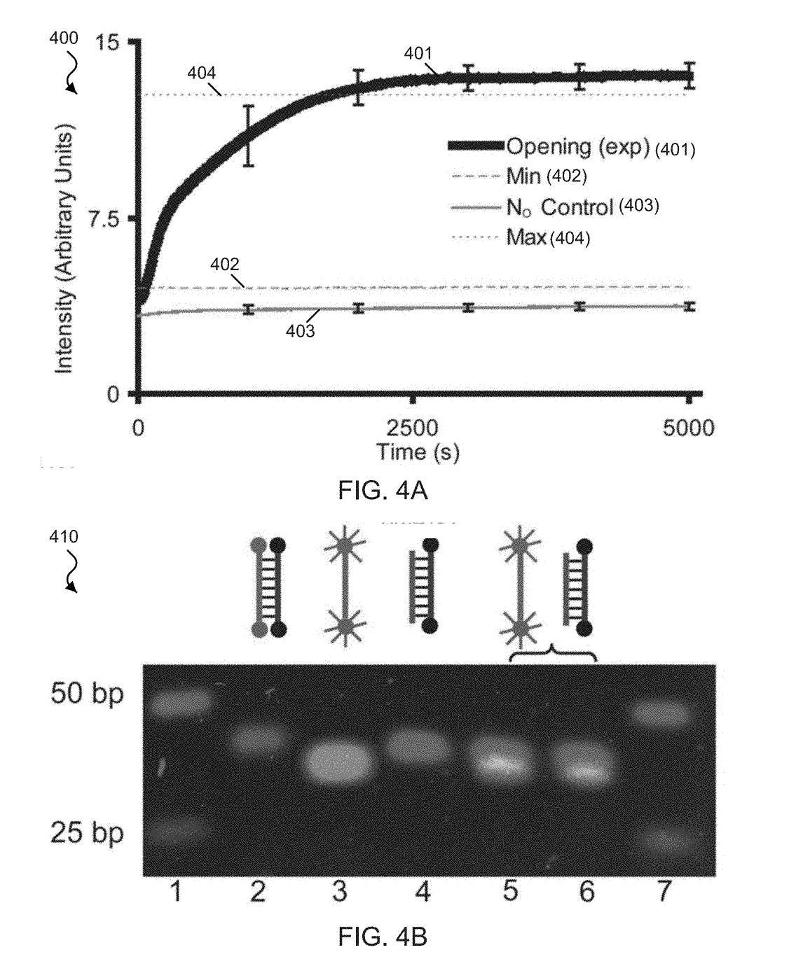

Exemplary implementations of the exemplary DNA zipper tweezers included performing fluorescence observation of the zipper tweezers activity. For example, FIG. 4A shows a fluorescence spectra plot 400 from the 6-carboxyfluorescein (FAM) fluorophore on the W strand, e.g., which was observed with an excitation/emission of 495 nm/520 nm. The spectra plot 400 includes an opening plot 401 displaying the time-lapsed fluorescence from the opening reaction of the exemplary zipper tweezers [N:W] that was observed immediately after initiation (e.g., t=0) from the addition of 10× more opening strands (O) than exemplary zipper tweezers, e.g., as described by the equation [N:W]+10O→[N:O]+W+9O. The spectra plot 400 includes a Min plot 402 that represents the initial basal fluorescence of the [N:W] helix prior to initiation of the reaction. The spectra plot 400 includes a Max plot 404 that represents the maximum fluoresce signal obtainable from the opening reaction. For example, the fluorescence from the thermally annealing of the opening reaction produced the idealized end product [N:O]+W+9O. The spectra plot 400 includes a N_{O} Control stability plot 403 that represents the measure of the rate of strand exchange between the normal N strand initially in the zipper [N:W] and 10×N_{O} (e.g., the N sequence without any quenchers) added at time (t=0) described by the steady state reaction [N:W]+10N_{O} ↔(1−a)[N:W]+a[N_{O}:W]+(9−a)N_{O}+aN, where a≤1. For example, time-lapse fluorescence of the initial zipper configuration [N:W] displayed a small but steady basal fluorescence, e.g., due to colocalization of fluorescent markers and quenchers, as shown by the Min plot 402 in the spectra plot 400.

For example, when O was added to the [N:W] helix, a continuous increase in fluorescence was determined, e.g., that stabilized to a final steady state as shown by the Opening plot 401 in the spectra plot 400. An increase in the fluorescence can be considered to be due to delocalization of the fluorophores and quenchers (e.g., separation of W from N). For example, completion of the reaction was confirmed by comparing the peak signal produced by the thermal annealing of [N:W] with O, e.g., producing the highest fluorescence and lowest energy configuration of the system, as shown by the Max plot 404 in the spectra plot 400. The exemplary results indicated that the zipper reaction was driven to its completion in about ˜42 min at 37° C. Table 3 presents the time required for 50% completion of zipper opening reactions (t_{1/2}) at 37° C.

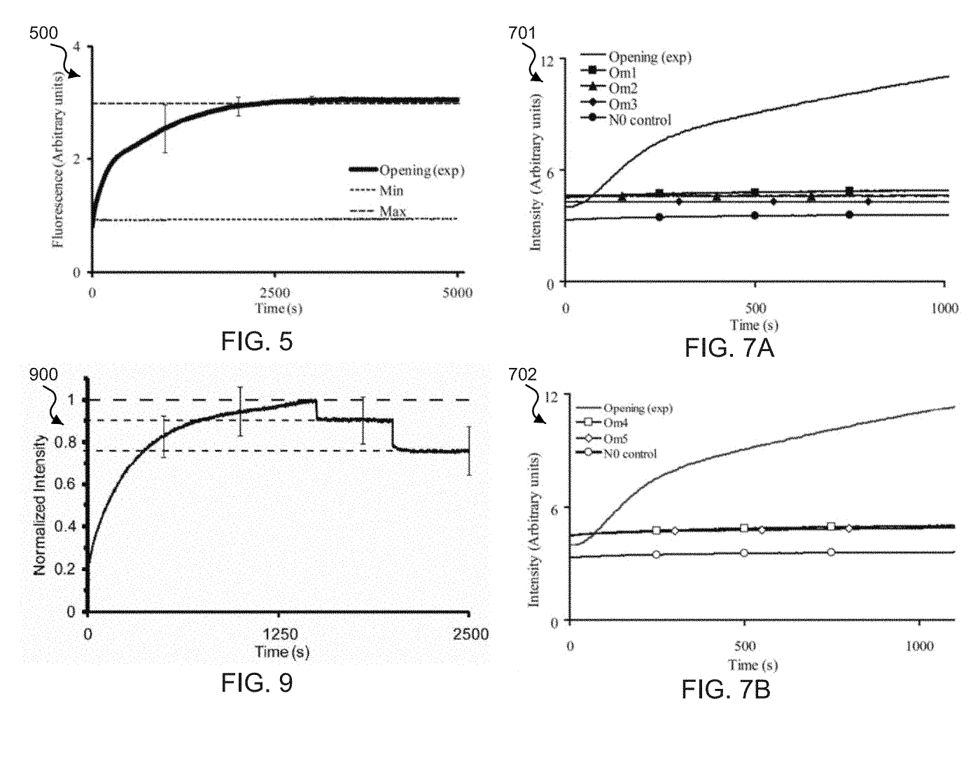

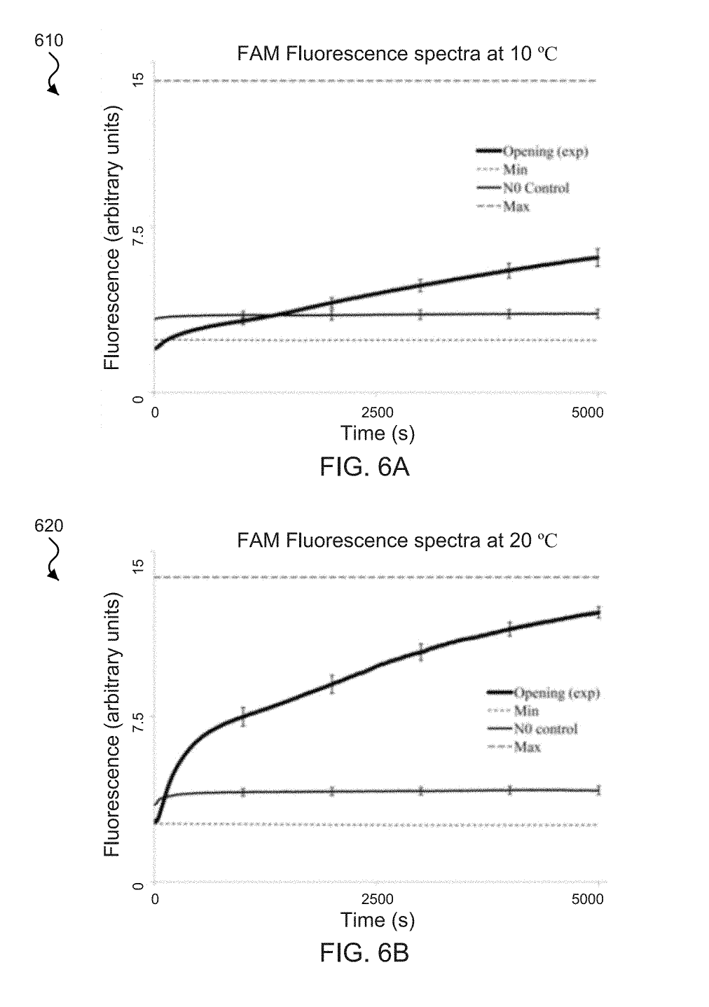

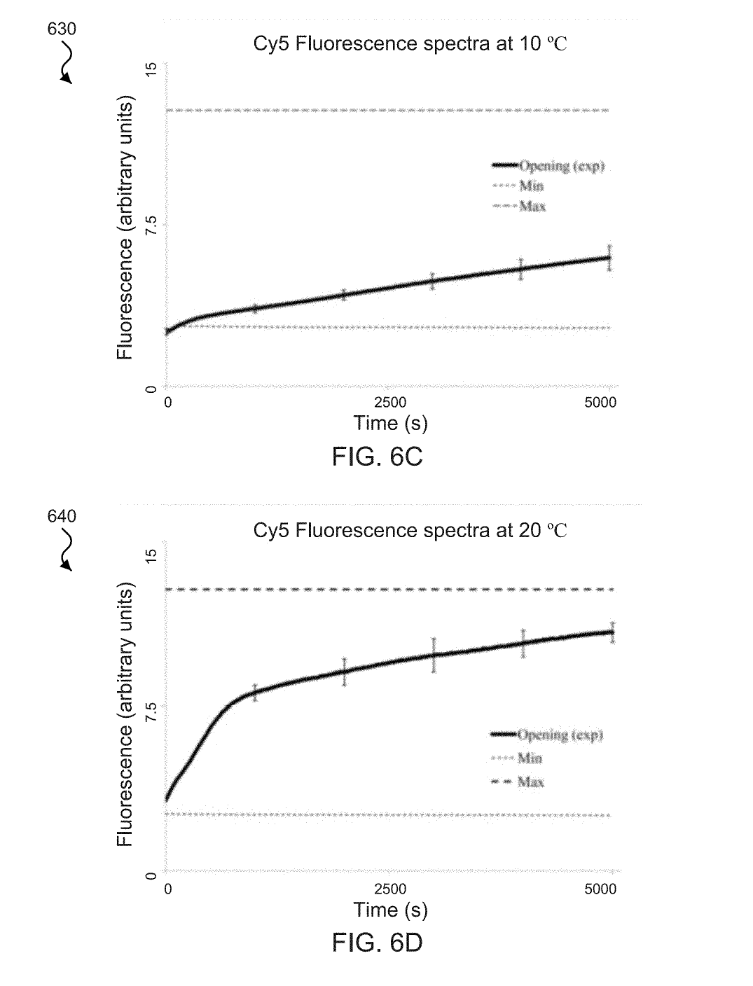

For example, in these exemplary implementations, the increase in fluorescence observed in the zipper reaction could also result from spontaneous strand dissociations, random base pair mismatches (e.g., resulting in the formation of overhangs), and slipping between the strands (e.g., resulting in delocalization of fluorescent probes, due to weaker interactions in [N:W] helix). For example, to rule out these possibilities, the [N:W] helix was probed by observing the change in basal fluorescence after adding a ten-fold higher concentration of No (e.g., 10×N_{O}, the N sequence without any quenchers). If any of the above possibilities should take place, then the formation of [N_{O}:W] would result in an increase in the fluorescence. Absence of any such increase can suggest that such possibilities are either absent or insignificant, e.g., as seen in FIG. 4A by the N_{O} Control stability plot 403. For example, FIG. 5 includes the fluorescence from Cy5 on the other end of W. In addition, for example, no significant change in the basal fluorescence was observed at 10° C. and 20° C., shown in FIGS. 6A-6D, which can also suggest that such possibilities are either absent or insignificant in the exemplary implementations of the disclosed zipper tweezers.

FIG. 5 shows a data plot 500 that demonstrates time lapse fluorescence spectra from the Cy5 fluorophore on the 3′ end of an exemplary W strand observed at 37° C. For example, the data plot 500 displays the fluorescence of the opening reaction of the zipper [N:W] examined immediately after the addition of 10 times O at (t=0). For example, the min dashed line represents the basal fluorescence of the [N:W] helix prior to initiation of the reaction. For example, the max dashed line represents the maximum fluorescence signal obtainable from the opening reaction. For example, the data plot 500 represents the fluorescence from the thermally annealed opening reaction producing the idealized end products [N:O]+W+9O.

FIGS. 6A-6D show fluorescence spectra plots of exemplary W strands functionalized with the FAM fluorophore on the 5′ end and the Cy5 fluorophore on the 3′ end of the W strand. FIG. 6A shows a spectra plot 610 showing the exemplary FAM fluorescence of the FAM-Cy5 functionalized W strands observed with excitation/emission of 495 nm/520 nm at 10° C. FIG. 6B shows a spectra plot 620 showing the exemplary FAM fluorescence of the FAM-Cy5 functionalized W strands observed with excitation/emission of 495 nm/520 nm at 20° C. FIG. 6C shows a spectra plot 630 showing the exemplary Cy5 fluorescence of the FAM-Cy5 functionalized W strands observed with excitation/emission of 648 nm/668 nm at 10° C. FIG. 6D shows a spectra plot 640 showing the exemplary Cy5 fluorescence of the FAM-Cy5 functionalized W strands observed with excitation/emission of 648 nm/668 nm at 20° C. For example, in the spectra plots 610, 620, 630 and 640, the opening plot displays the time-lapsed fluorescence from the opening reaction of the zipper [N:W], e.g., observed immediately after initiation (t=0) from the addition of 10× more O than zipper described by [N:W]+100→[N:O]+W+9O. The Min plot displays the initial basal fluorescence of the [N:W] helix, e.g., prior to initiation of the reaction. The Max plot represents the maximum fluoresce signal obtainable from the opening reaction. For example, the fluorescence from the thermally annealing the opening reaction produced the idealized end product [N:O]+W+9O. The N_{O} Control stability plot represents the measure of the rate of strand exchange between the normal N strand initially in the zipper [N:W] and 10×N_{O} (e.g., the N sequence without any quenchers), e.g., added at time (t=0) described by the steady state reaction [N:W]+10N_{O} ↔(1−a)[N:W]+a(N_{O}:W)+(9−a)N_{O}+aN, where a≤1.

Exemplary implementations were also performed to probe the specificity and efficiency of zipper action for seven different opening strands with significant (e.g., 16-24%) sequence mismatches O_{M1}-O_{M7}, shown in Table 1, measured at 37° C. Exemplary results are shown in FIGS. 7A and 7B. The exemplary data suggested that the zippers have a relatively high degree of binding specificity to the opening strands. For example, the zippers remained relatively stable after the addition of opening strands that contained, for example, 6-9 base pair mismatches (as shown in Table 1) distributed along their length.

FIGS. 7A and 7B show exemplary data plots that demonstrate time-lapse fluorescence of FAM-tagged zipper tweezers, e.g., tagged at the 5′ end of an exemplary W strand. The exemplary data plots include opening strands O_{M1}-O_{M5}, which contain 6-9 mismatched (e.g., sequences shown in Table 1). Data plot 701 shown in FIG. 7A includes opening strands O_{M1}-O_{M3}, and data plot 702 shown in FIG. 7B includes opening strands O_{M4}-O_{M5}. The exemplary opening plots O, or O_{M1}-O_{M5}, display the time-lapsed fluorescence of the opening reaction of the exemplary zipper tweezers [N:W] examined immediately after initiation (t=0) from the addition of 10 times O, or O_{M1}-O_{M5} than the [N:W] helix.

Exemplary implementations of the exemplary DNA zipper tweezers included performing DNA gel electrophoresis of the zipper tweezers action. For example, the zipper action was validated using fluorescent gel imaging, and the products and reactants of the zipper reaction along with thermally annealed sample [N:O] as a control were analyzed. For example, since the mass-charge ratio of double- and single-stranded DNA is the same in the exemplary implementations, the exemplary products and reactants ran collinear on the gel electrophoresis. For example, the double strands were identified with Ethidium Bromide (EtBr), and the single strands were identified with fluorophores. FIG. 4B shows the exemplary findings of fluorescence observation of the zipper action.

FIG. 4B shows exemplary gel electrophoresis data 450 showing the position of dsDNA in the gel determined by EtBr staining (shown in RED) and the position of the single-stranded W strand in the gel determined by Cy5 staining (shown in GREEN). For example, the exemplary W strand allowed its position to be recorded only when single-stranded because the W strand is quenched by the Iowa Black quencher when coupled to an N strand. The exemplary contents of the six lanes between the two 25 nt DNA step ladders on the gel, shown from left to right, are as follows. Lane (1) shows the initial zipper helix in its quenched state [N:W]. Lane(2) shows single-stranded W with attached Cy5 fluorophore. Lane (3) shows the resulting helix after opening of the zipper [N:O]. Lane (4) shows the opened zipper, e.g., after 2 hr of the exemplary reaction: [N:W]+10O→[N:O]+W+9O. Lane (5) shows the exemplary reaction after thermally annealing, e.g., which produces the lowest energy state of the system and the maximum fluorescence signal possible from the reaction [N:W]+10O→[N:O]+W+9O. Lane (6) shows the exemplary thermally annealing control.

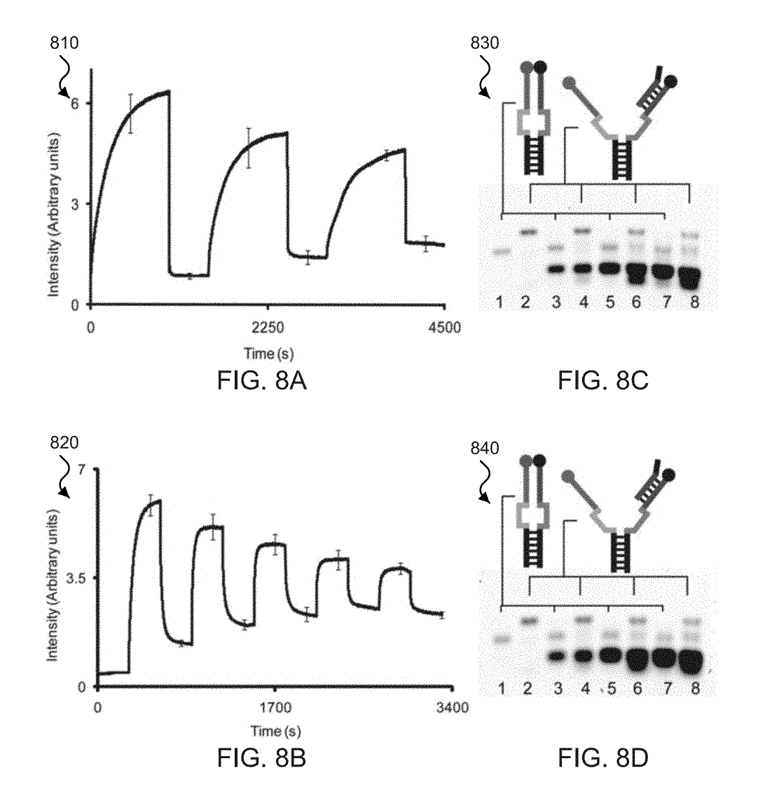

Exemplary implementations of the exemplary DNA zipper tweezers included characterizing the zipper tweezers activity. For example, the activity of the exemplary DNA zipper tweezers was examined by tagging the W strands with Cy5.5; the N strands with Iowa Black RQ; and both opening and closing strands without fluorophores. Exemplary time lapse fluorescence measurements and fluorescence images from DNA gel electrophoresis from three successive opening and closing cycles of the disclosed DNA zipper tweezers using the O₁, C_{1-LNA} pair are shown in FIG. 8A. For example, the reaction is illustratively shown in FIG. 3 and can be summarized in Eq. (4) and Eq. (5) as:

[W_{Z}:N_{Z}]+O₁→W_{Z}+[N_{Z}:O₁] (4)

W_{Z}+[N_{Z}:O₁]+C₁→[O₁:C₁]+[W_{Z}:N_{Z}] (5)

FIGS. 8A-8D show exemplary opening and closing cycling data of exemplary zipper tweezers using an exemplary opening strand O₁ and an exemplary closing strand C_{1-LNA}. For example, the opening strand O₁ opened the exemplary zipper tweezers using the disclosed zipper mechanism, and C_{1-LNA} closed the tweezers, e.g., by hybridizing to O₁ facilitated by a 7 nt overhang. For example, FIG. 8A shows an exemplary time-lapsed fluorescence spectra plot 810 showing three successive opening and closing cycles of the disclosed DNA zipper tweezers. For example, initially the exemplary zipper tweezers is configured in the closed position [W_{Z}:N_{Z}](e.g., with concentration of 1×) before the addition of an opening strand O₁. For example, since the quencher and Cy5.5 are co-localized, there is no significant fluorescence. For example, after the addition of 10×O₁ (e.g., as shown during the exemplary 0-1000 s interval), the exemplary zipper tweezers can switch to the hold position [N_{Z}:O₁], e.g., where the fluorescence from Cy5.5 can almost immediately begin to rise. The increasing fluorescence signals can be seen in the plot 810 from 0 to 1000 s, 1500 to 2500 s, and 3000-4000 s. For example, immediately after the addition of 20×C_{1-LNA} (e.g., as shown during the exemplary 1000-1500 s interval), the exemplary zipper tweezers switches to release position [O₁:C_{1-LNA}], e.g., C_{1-LNA} hybridizes to O₁, the waste product [O₁:C_{1-LNA}] is released, and the exemplary zipper tweezers close. Also, this release resets the exemplary zipper tweezers back to the closed position [W_{Z}:N_{Z}], and the fluorescence signal rapidly decreases. The decreasing fluorescence signal can be seen in the plot 810 from 1000-1500 s, 2500-3000 s, and 4000-4500 s. Exemplary remaining cycles were conducted by adding 30×, 50×O₁ and 40×, 60×C_{1-LNA} respectively.

For example, the exemplary O₁ strand contained 28 nt and was configured to be complementary to N_{Z} (21 nt), e.g., the additional 7 nt formed a DNA overhang, which enabled the exemplary O₁ strand to be removed by the exemplary C_{1-LNA} strand. The exemplary C_{1-LNA} strand had 21 nt and contained six LNA base modifications (as shown in Table 2). For example, the exemplary C_{1-LNA} strand was configured to be complementary to the entire 7 nt overhang of the exemplary O₁ strand and its remaining 14 nt. For example, since the exemplary C_{1-LNA} strand and the exemplary W_{Z} strand are complements (as shown in Table 2), the exemplary C_{1-LNA} strand was made shorter than the exemplary O₁ strand to reduce the affinity between them. For example, this can necessitate the condition that the Tₘ of [W_{Z}:C_{1-LNA}] be sufficiently less than the operating temperature of the exemplary zipper tweezers. Otherwise, the exemplary W_{Z} strand can hybridize with the C_{1-LNA} strand, e.g., preventing the exemplary zipper tweezers from closing [W_{Z}:C_{1-LNA}]. The six exemplary LNA bases were positioned near the overhang binding end of the C_{1-LNA} strand in order to preferentially increase the binding affinity between the C_{1-LNA} strand and the O₁ strand.

For example, to examine the robustness of the exemplary zipper tweezers, they were driven further for three opening/closing cycles (as shown in the plot 810 in FIG. 8A), e.g., by adding O₁ and C_{1-LNA}. The exemplary data show a strong robustness; for example, the exemplary zipper tweezers cycled efficiently among the closed, capture, release, and back to closed positions. Exemplary peak fluorescence data from each of the successive opening cycles, however, can be seen to decrease relative to the prior peaks. For example, this can be considered due to dilution of the sample by the addition of the opening and closing strands (e.g., 10 μL each) at each step. For the demonstration of this effect, a time lapse fluorescence measurement from a dilution control sample is shown in FIG. 9.

FIG. 9 shows a data plot 900 of the normalized fluorescence spectra from an exemplary opened zipper tweezers. The exemplary data shown in the data plot 900 demonstrates the effect of sample dilution on the fluorescence signal intensity. For example, 10 μL of buffer was successively added to a cuvette with 100 μL of sample in 40 μL sampling window to measure the change in signal with the addition of solution. As shown in the data plot 900, the top dashed line represents 100% signal intensity. The lower dashed line represents 90% of the original signal intensity, which shows a linearly dependent signal intensity after a ˜10% dilution. The lowest dashed line represents ˜75% of the original signal intensity, which shows the signal intensity after the addition of 20 μL.

It is noted, for example, that as the peaks shown in the plot 810 in FIG. 8A decreased from the dilution effects, the minimum fluorescence from the closed tweezers was expected to remain the same or to decrease as well. However, as shown in the plot 810, the minimum fluorescence increased during these cycles. For example, elevated basal fluorescence with successive cycles may result from increased competition from the waste products. For example, that after completion of the exemplary three cycles, there are 90 times more opening strands and 120 times more closing strands present in the solution than the exemplary zipper tweezers.

For example, to confirm that the loss of functionality was due to the excess waste product and not from the destruction of the exemplary zipper tweezers, exemplary reactions from four successive opening and closing cycles were subjected to DNA gel electrophoresis. For example, FIGS. 8C and 8D shows the DNA electrophoresis gel data 830 and 840 that demonstrates the products from two opening/closing cycles of the zipper tweezers that were imaged using EtBr staining (shown in GREEN) and the fluorescence from the Cy5.5 fluorophore (shown in RED) attached to the W_{Z} end of the zipper tweezers. Exemplary lanes (1 and 7) contained a 25 nt DNA step ladder. Exemplary lanes (2, 4, and 6) contained the closed tweezers (e.g., quenched). Exemplary lanes (3 and 5) contained the open tweezers (e.g., fluorescent). As shown in FIGS. 8C and 8D, exemplary purple bands represent the result of co-localization of the EtBr and Cy5.5 signals, and the large red bands at the bottom of lanes (4, 5, and 6) represent excess double helices waste product from the reversing of the tweezers. For example, to rule out dilution effects, the concentrations of exemplary zipper tweezers in each cycle were kept the same. The exemplary gel data 830 and 840 show that the opening efficiency of the gates reduces with successive cycles. For example, there was no visible difference between the gel containing exemplary zipper tweezers (e.g., gel data 830) and thermally annealed control (e.g., gel data 840). For example, if the zipper tweezers were to fail, the zipper tweezers would be expected to come apart at the lower hinge holding the two sides of the device together, but this portion was shown to be relatively stable and has a calculated Tₘ of ˜67° C. For example, if the tweezers did dissociate with successive cycles, then thermal annealing would heal the system, and that would be revealed as a visible difference in the gel data. The exemplary data in FIGS. 8C and 8D show that the robustness of the disclosed zipper tweezers is maintained.

Exemplary implementations of the exemplary DNA zipper tweezers included characterizing zipper tweezers kinetics, and for example, the role of overhangs and locked nucleic acid (LNA) bases. LNA bases are known to be highly selective and capable of single nucleotide discrimination when hybridizing and have increased target specificity. The exemplary results shown in FIG. 8A indicates that the exemplary zipper tweezers closed about 10 times faster than it opened. For example, the exemplary opening strand O₁ alone opened the zipper tweezers using the disclosed zipper mechanism, and the exemplary opening strand C_{1-LNA} removed the O₁ strand, e.g., by taking advantage of a 7 nt overhang on the O₁ strand. To investigate the opening rates of the tweezers using the zipper mechanism together with an overhang, an exemplary opening strand O₂ was configured. For example, the exemplary opening strand O₂ bound to 7 nucleotide units of the N_{L} strand and 14 nucleotide units of the N_{Z} strand. The exemplary 02 strand also contained a 7 nt overhang to facilitate its removal by an exemplary closing strand C₂. For example, the combination of the two overhangs can allow the zipper tweezers to be cycled more quickly. For example, using the O₂ strand and C₂ strand pair, the zipper tweezers was cycled five times in ˜600 s shown in FIG. 8B, as compared to ˜1200 s for the O₁ strand and C_{1-LNA} strand pair shown in FIG. 8A.

FIG. 8B shows an exemplary time-lapsed fluorescence spectra plot 820 showing five successive opening and closing cycles of the disclosed DNA zipper tweezers. For example, initially the exemplary zipper tweezers is configured in the closed position [W_{Z}:N_{Z}] (e.g., with concentration of 1×) before the addition of the opening strand O₂, and subsequently the addition of a closing strand C₂. For example, the exemplary opening strand O₂ hybridized to 7 nt of an exemplary W_{L} strand, e.g., to speed up the opening of the zipper tweezers, and the exemplary closing strand C₂ hybridized to 7 nt of an overhang on the exemplary 02 strand.

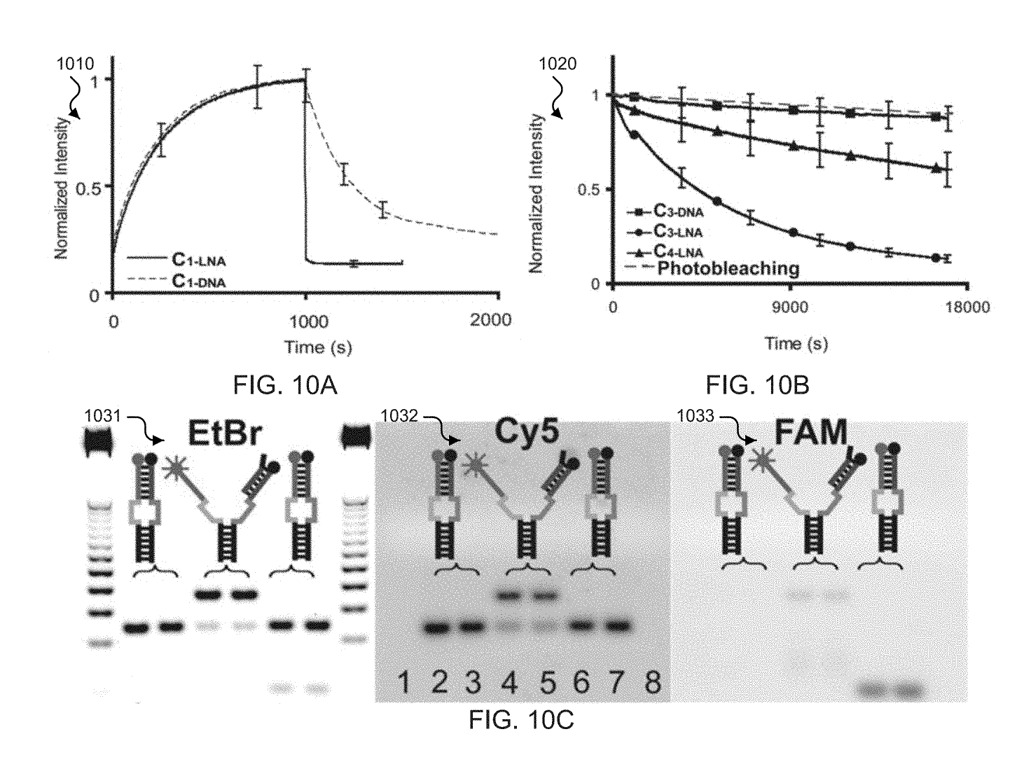

For example, the closing rates of the zipper tweezers were examined using the exemplary O₁ strand and the exemplary closing C_{1-LNA} implemented to obtain the opening/closing cycling data of the plot 810 in FIG. 8A compared to the exemplary 02 strand and the exemplary closing C₂ implemented to obtain the opening/closing cycling data of the plot 820 in FIG. 8B. The exemplary comparative data indicated that the C₁ strand removed the O₁ strand considerably faster as compared to the rate at which the C₂ strand removed the O₂ strand. For example, despite some subtle differences between the modes of operation using C_{1-LNA} and C₂, the major difference is the 6 LNA base modifications concentrated at the overhang portion of the C_{1-LNA} strand. For example, the zipper tweezers were examined by opening using the O₁ strand and closing with a C_{1-DNA} strand, e.g., a natural DNA strand with the identical sequence as C_{1-LNA} to assess the effect of LNA, as shown in FIG. 10A.

FIG. 10A shows a normalized fluorescent spectra plot 1010 comparing closing kinetics of exemplary zipper tweezers using the exemplary C₁ and C_{1-DNA} strands after opening with the O₁ strand. Both the C₁ and C_{1-DNA} strands have identical base pair sequences, except the C₁ strand contains LNA bases and C_{1-DNA} does not. Some exemplary possible factors responsible for increasing the closing rate of tweezers when the 6 LNA bases are added to the DNA sequence can include the increased hybridization energy between an LNA/DNA helix, the structural conformation of the C₁ strand enabling it to hybridize to the overhang more quickly, and/or the LNA bases lowering the binding affinity of the C₁ strand to the W_{Z} strand.

For example, zipper tweezers with overhangs can be more prone to random hybridizations. In these situations in which overhangs are undesirable, LNAs can be employed. For example, LNA/DNA helices have higher Tₘ than DNA/DNA helices for a given sequence, and this energy difference can be used to invade small DNA duplex. However, such reactions can be relatively slow. For example, one such system is demonstrated with the O₃ opening strand and the C_{3-LNA} closing strand, as shown in FIG. 10B.

FIG. 10B shows a normalized fluorescent spectra plot 1020 comparing closing kinetics of exemplary zipper tweezers using LNA closing strands, e.g., to invade the duplex formed by [N_{Z}:O₃] after opening the zipper tweezers with the opening O₃ strand. For example, the three exemplary closing strands C_{3-LNA}, C_{4-LNA} and C_{3-DNA} have identical base pair sequences, except C_{3-LNA} and C_{4-LNA} contain LNA bases. For example, the C_{3-LNA} strand contains 7 LNA bases concentrated in the N_{L} binding portion. For example, the C_{4-LNA} strand contains 8 LNA bases distributed evenly across its length. The exemplary C_{4-LNA} strand can close the tweezers slower because it has a higher affinity for the W_{Z} strand part of the exemplary zipper tweezers. For example, the C_{3-DNA} strand does not contain any LNA bases and was included in the exemplary implementations as a stability control, e.g., to measure the rate of spontaneous disassociation. The exemplary O₃ strand contained only natural bases and it did not contain any overhangs to facilitate its removal. Exemplary binding interactions of the strands were as follows. The exemplary O₃ strand hybridized with lower 14 nt of N_{Z} and to the first 10 nt of the loop. The exemplary C_{3-LNA} strand was complementary to the exemplary O₃ strand and contained seven LNA modifications, e.g., most of which were positioned in the loop binding portion. As shown in FIG. 10B, the O₃ strand and C_{3-LNA} strand pair opened the tweezers in less than 300 s and closed it in about 18000 s (5 h). For example, a control closing strand C_{3-DNA} containing identical sequence as C_{3-LNA}, but only natural DNA bases, was implemented, but did not reclose the tweezers. For example, the plot 1020 includes decay in the signal, which can be attributed to photobleaching of the sample.

In another example, an exemplary closing strand C_{4-LNA} was configured to have the same base pair sequence as C_{3-LNA} containing 8 LNA modifications evenly distributed along its length. For example, the even distribution of the LNA modifications along the C_{4-LNA} strand resulted in a significant decrease in the opening rate of the zipper tweezers (˜3 times). This exemplary decreased opening rate may be caused by a higher affinity between C_{4-LNA} and the W_{Z} portion of the zipper tweezers (e.g., because the LNA bases are positioned along the section that is complementary to W_{Z}). The disclosed DNA based nanomachines can be configured without overhangs to achieve rapid open/close cycling functionality, e.g., by using locked nucleic acids (LNAs) and peptide nucleic acids (PNAs) together with the exemplary zipper tweezers.

Exemplary examinations into different zipper tweezers states and actions were performed by fluorescent DNA gel electrophoresis. FIG. 10C and the results verify their different states namely, close, hold & capture, release and close positions for a particular set of 03 and C_{3-LNA} strands.

FIG. 10C shows DNA electophorisis gel images 1031, 1032, and 1033 of exemplary zipper tweezers opened using the exemplary O_{3-FAM} strand (e.g., the O₃ sequence with a FAM fluorophore on the 5′ end), followed by closing with the exemplary C_{3-LNA} strand. The gel images 1031, 1032, and 1033 verified that O_{3-FAM} hybridized to the exemplary zipper tweezers and that C_{3-LNA} hybridized to O_{3-FAM}. For example, lanes (1 and 8) contained a 25 nt DNA step ladder; lanes (2 and 3) contained the closed tweezers; lanes (4 and 5) contained the tweezers opened by O_{3-FAM}; and lanes (6 and 7) contained the gates closed by C_{3-LNA}. In the exemplary implementation, the tweezers they were opened using only 80% of O_{3-FAM} required to open all of the zipper tweezers. The exemplary results included faint bands (e.g., shown in lanes (4 and 5) below the open tweezers. The exemplary zipper tweezers included a Cy5.5 on the N_{Z} strand and without a quencher on the W_{Z} strand. Thus, 20% of the tweezers remained closed and fluorecsent in lanes (4 and 5).

Exemplary opening schemes (e.g., zipper alone and N_{L} hybridizing overhang) and exemplary different closing schemes (e.g., overhang, overhang with LNAs, and LNAs only) are described for implementing the disclosed zipper tweezers of the disclosed technology. For comparing their kinetics, time required for the 50% completion of the opening and closing reaction (t_{1/2}) with different strand configurations are shown in Tables 3 and 4, respectively.

Exemplary techniques and principles for creating the disclosed molecular zipper-based devices and systems include engineering the functional zipper with regards to the total driving energy and how this energy is distributed along the length of the strands. For example, the nucleotide units (e.g., nucleobases) providing the driving energy must occur with a sufficient frequency along the length of the weak strand in order for a favorable displacement reaction by a target strand. For example, if too many natural DNA bases occur between the driving bases (e.g., inosine), the reaction may terminate. The entropy-induced statistical fluctuations between the bases can enable the reaction to progress along sufficiently small sections of natural base pairs. For example, the length of the natural section that could be overcome by the statistical fluctuations is a temperature- and sequence-dependent property. Also, for example, the bases used to supply the driving energy need not be inosine, as other synthetic bases can be used (e.g., in an engineered strand) that hybridize with less or more than natural affinity. For example, FIGS. 1A and 1B show other non-naturally-occurring nucleobases configured in a passive strand.

Exemplary techniques and principles for creating the disclosed molecular zipper-based devices and systems include engineering the functional zipper with regards to the cross-binding nature of the closing strands. For example, a difference between the energies of the hybridization of [Cᵢ:W_{Z}] and [Cᵢ:Oᵢ] can be incorporated into the configuration of the molecular zipper-based devices and systems. For example, a temperature window can be incorporated in which the zipper tweezers can function, e.g., an operating temperature of the tweezers can be significantly chosen below the Tₘ of the zipper portions of the tweezers (e.g., [W_{Z}:N_{Z}]) and significantly above the Tₘ of [Cᵢ:W_{Z}]. Exemplary implementations of the disclosed technology demonstrated the increase of the operating temperature range of the disclosed zipper tweezers, e.g., by DNA overhangs, truncating the length of Cᵢ relative to Oᵢ, and using LNA base modifications concentrated at sequence portions that are uncommon between Cᵢ and W_{Z}. For example, DNA strands naturally self-assemble into energetically stable configurations. The disclosed technology can control the interaction energies of the systems constituents to minimize unwanted self-assembly from DNA. For example, if semi-stable unwanted hybridization between the different system elements occurs, it can significantly affect the kinetics of the system, and if stable hybridizations occur (unwanted self-assembly), the function of the system can completely cease.

The disclosed molecular zipper-based tweezers include a variety of advantages, e.g., including having a driving energy that is distributed over the entire length of the fuel strands, which allows more driving energy to be employed. Exemplary molecular zipper-based tweezers devices can sense and capture longer DNA strands with additional abilities to tune the kinetics (e.g., open/close mechanisms) as compared to non-zipper-based tweezers that contain all of their driving energy at short overhangs or loops. Exemplary molecular zipper-based tweezers devices can also allow for the use of longer fuel strands, e.g., because the disclosed zipper tweezers do not have sticky ssDNA overhangs that protrude from the ends of the tweezers in the sensing (e.g., closed or zipped) position. This can enable the exemplary molecular zipper-based tweezers devices to be opened without the use of overhangs, e.g., which can allow spontaneous regeneration to its closed position.

In another aspect, the disclosed technology can include devices, systems, and techniques that can provide a nanoscale molecular-based actuator, e.g., molecular zipper based springs. For example, the exemplary molecular zipper based springs can contract and impart force. For example, the molecular zipper based springs that can be implemented in applications that require tools that are small and sensitive enough to interact with molecules of interest, e.g., including smart drug carriers, sensors and devices for nanoscale transport and manipulation of biological macromolecules. DNA can be employed in the molecular zipper based springs of the disclosed technology, e.g., which can offer innate self-assembly properties, flexibility in design of secondary structures, and desirable length scale. In some examples, a DNA zipper based spring can include an inosine-based zipper mechanism at its functional core in which an inosine-containing strand creates a weak complement to a natural DNA strand.

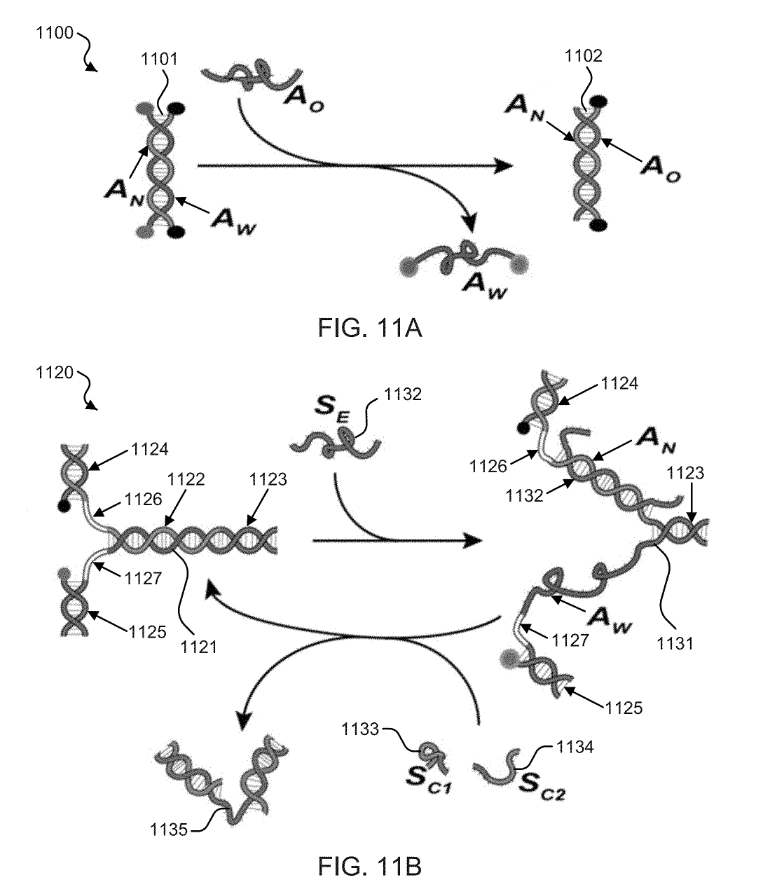

FIG. 11A shows an exemplary schematic illustration 1100 of an exemplary molecular zipper mechanism, e.g., configured as a part of a DNA based zipper spring actuator device. An exemplary molecular zipper structure 1101 can include a double-stranded helix including a normal strand (A_{N}), e.g., containing naturally-occurring bases, coupled to a weak strand (A_{W}), e.g., containing non-naturally-occurring bases such as inosine (I) substituted for guanine (G). For example, by altering the number and spacing of the inosines, A_{W} can be engineered to provide less-than-natural bonding affinities to A_{N}, e.g., resulting in a weaker bond. Thus, A_{W} can be a complement to A_{N} with less hybridization energy than, for example, a natural ssDNA. As shown in FIG. 11A, an opening fuel strand (A_{O}), e.g., configured as a natural complement of A_{N}, can be introduced to the exemplary zipper system and can competitively displace A_{W} from the zipper duplex [A_{W}:A_{N}], e.g., forming the energetically more stable helix [A_{O}:A_{N}] represented by molecular zipper structure 1102.

FIG. 11B shows an exemplary schematic illustration 1120 of an exemplary molecular zipper based spring device, e.g., a DNA based zipper spring actuator device. An exemplary contracted DNA based zipper spring 1121 can include a double-stranded DNA molecule that can include multiple segmented members. For example, the contracted DNA based zipper spring 1121 can include a zipper member 1122 connected to a hinge member 1123. The zipper member 1122 can be held together at one end by the hinge member 1123. The exemplary zipper member 1122 can include a normal strand (A_{N}), e.g., containing naturally-occurring bases, coupled to a weak strand (A_{W}), e.g., containing non-naturally-occurring bases such as inosine (I) substituted for guanine (G), as shown in the molecular zipper structure 1101 of FIG. 11A. The exemplary hinge member 1123 can include a region of the double-stranded DNA molecule that includes hybridized strands of nucleotide units having naturally-occurring bases on each strand configured in a complementary sequence with one another, e.g., and therefore tightly coupled. For example, when the zipper spring is contracted, the two complementary zipper portions of the springs A_{W} and A_{N} are hybridized together (e.g., [A_{W}:A_{N}]). The hinge member 1123 can hold the two strands of the zipper member 1122 together (and thereby hold the zipper spring together) when the zipper spring is extended. The contracted DNA based zipper spring 1121 can also include an arm member 1124 (e.g., also referred to as the B strand) branched from the A_{N} strand of the zipper member 1122 and an arm member 1125 (e.g., also referred to as the L strand) branched from the A_{W} strand of the zipper member 1122. For example, the branched connection between the arm member 1124 and the A_{N} strand can include a toehold member 1126 configured to a particular length, e.g., comprising a particular number of nucleotide units. The branched connection between the arm member 1125 and the A_{W} strand can include a toehold member 1127 configured to a particular length, e.g., comprising a particular number of nucleotide units, which can be configured to match the length of toehold member 1126. For example, the toehold members 1126 and 1127 can be used to extend the zipper springs faster than the zipper mechanism can without the exemplary toehold members. The exemplary toehold members 1126 and 1127 can be configured to be a 6 nt toehold, e.g., depicted by the white piping between the arm member 1124 and the A_{N} strand of the zipper member 1122. For example, the arm members 1124 and 1125 can contain fluorescent labels (e.g., fluorophores functionalized to an end of the arm members), which can allow determination and/or monitoring of the zipper spring's contraction or extension functionalities.

The exemplary schematic illustration 1120 shows the opening of the exemplary zipper spring using the disclosed zipper mechanism. An exemplary extended DNA based zipper spring 1131 is shown in an extended position, which includes the two zipper strands A_{N} and A_{W} separated, e.g., by uncoupling the hybridized complementary nucleobases between the A_{N} and A_{W} strands to an unzipped or open position. For example, the exemplary extended DNA based zipper spring 1131 can be unzipped to an extended position by a target molecule that includes an extending strand 1132 (e.g., also referred to as an S_{E} strand) which can hybridize to the A_{N} strand of the zipper member 1122, thereby displacing A_{W} from A_{N}. The extending strands 1132 (S_{E}) can be configured as an opening fuel strand (A_{O}) with toeholds on either end or both ends, e.g., to assist in contraction and extension of the zipper springs. For example, when the S_{E} extending strand 1132 was introduced to the contracted spring (e.g., the contracted DNA based zipper spring 1121), the S_{E} extending strand 1132 hybridizes to the A_{N} portion of the zipper member 1122 by competitively displacing A_{W} away from A_{N} using the zipper process causing the zipper spring to extend (e.g., into the extended DNA based zipper spring 1131). For example, the displacement reaction occurs because the enthalpies of the C≡G bonds between S_{E} and A_{N} are stronger by ˜8 kJ/mol than those of the I═C bonds between A_{W} and A_{N}.

Once the exemplary zipper springs have been extended by the S_{E} extending strand 1132, the exemplary extended DNA based zipper spring 1131 can once again be reset (e.g., contracted) by introducing contracting fuel stands 1333 and 1334 (e.g., also represented as an S_{C1} strand and an S_{C2} strand, respectively). For example, the S_{E} extending strand 1132 that is bound to the A_{N} strand of the zipper member 1122 on the extended DNA based zipper spring 1131 can be removed by the contracting strands 1333 and 1334 and the A_{W} and A_{N} portions can re-hybridize together, e.g., resetting the zipper spring back to the contracted state. For example, the S_{C1} and S_{C2} contracting fuel strands 1333 and 1334 can remove the S_{E} extending strand 1132 by hybridizing to exemplary toehold nucleotide units (e.g., 12 nt toeholds) on the S_{E} extending strand 1132 and subsequently to bases of the zipper-hybridizing portion on the S_{E} extending strand 1132. In some examples, the three strands (e.g., S_{E}, S_{C1} and S_{C2}) form a waste product 1135, which can drift away and leave the exemplary zipper springs to re-hybridize and contract. For example, the two strands S_{C1} and S_{C2} can remove the S_{E} strand from the A_{N} portion of the zipper spring because there is additional energy in the exemplary toeholds (e.g., 12 nt toehold) of S_{C1} and S_{C2} driving them to hybridize with the complementary 12 nt toehold on the S_{E} strand. For example, at 37° C. there is considerable amount of free energy (e.g., ΔG₃₇=−91.46 kJ/mol), e.g., favoring the S_{E} strand to extend the contracted zipper spring; and once the S_{E} strand is removed, there is also a considerable amount of free energy favoring the zipper spring to contract (e.g., ΔG₃₇=−87.90 kJ/mol).

Exemplary implementations were performed to demonstrate the described functionalities and capabilities of the disclosed molecular zipper tweezers. Chemicals and buffer solutions used in exemplary implementations were obtained from Sigma Aldrich (Saint Louis, Mo.) unless otherwise specified. The exemplary DNA constructs were obtained from IDT (Coreville, Iowa); the exemplary DNA ladders were obtained from Promega (Madison, Wis.); and the exemplary DNA gels were obtained from Lonza (Walkersville, Md.). Exemplary DNA constructs were suspended in DNAase-free 30 mM Tris and 0.16 M NaCl buffer solution pH 8.0.

Exemplary time-lapse fluorescence measurements of the exemplary zipper actions of exemplary zipper springs were visualized, for example, by tagging the strands with fluorescent probes (shown in Table 6) and observing the change in fluorescence with time using appropriate excitation (Ex) and emission (Em) wavelengths for the fluorophores. Exemplary Ex/Em conditions of FAM, Cy5 and Cy3 were observed at 495/520, 550/564 and 648/668 nm, respectively. Exemplary fluorescence measurements were conducted using a Perkin Elmer LS-50B luminescence spectrometer. Exemplary measurements were performed at 37° C. using quartz cuvettes with a 40 μL sampling volume (e.g., Sterna Cell 16.40F-Q-10/Z15) filled with 100 μL of sample at the start of each experimental implementation. The exemplary basal fluorescence of the quenched zipper was measured on each sample prior to data collection. For example, data was collected every 5 seconds. Each exemplary experimental implementation was repeated at least three times, e.g., to obtain an average. Exemplary error bars depict standard error of the mean, which are included in some of the exemplary data plots in the patent document. For example, the addition of exemplary fuel or anti-fuel strands included pausing measurements, e.g., for approximately 20 seconds.

Exemplary gel electrophoresis and fluorescence imaging analyses were performed in the exemplary implementations. For example, DNA gel electrophoresis was performed with 4% agarose gel at 5 V/cm in TBE buffer while monitoring the solution temperature to be less than 20° C. Exemplary reactions were incubated at 37° C. for at least 2 hours prior to gel examination. For example, each constituent of the gel was run in duplicate with a 25 base pair DNA ladder in the first and last lanes. Exemplary extension reactions were conducted, e.g., by adding ten times more extending strands than springs, and exemplary contractions reactions were conducted, e.g., by adding 20 times more contracting strands than springs to over saturate the existing extending strands. Exemplary reactants and controls were thermally annealed with equal concentrations of its components. For example, in order to observe single and double stranded DNA, positions of the strands within the gel were determined using fluorescent gel imaging and Ethidium Bromide (EtBr) staining. Exemplary gels were imaged with a Bio-Rad FX-Imager Pro Plus (Bio-Rad, Hercules, Calif.) and analyzed with the Quantity One software package (Bio-Rad). Modifications to the original gel images included brightness, contrast, cropping of the image area, over laying lines for reference and symbols for identification of the components. Exemplary Cy3 and EtBr imaging was performed with the internal 532 nm laser and 555 nm band pass filter, while exemplary Cy5 imaging uses an external 632 nm helium neon laser and a Newport 670 nm band pass fluorescence filter. Exemplary FAM imaging is performed using a 20 mW argon ion laser and a 530 nm band pass filter.

Exemplary fluorescence measurements and monitoring of the zipper springs were performed in the exemplary implementations. For example, time-lapsed fluorescence measurements of the zipper springs were performed using a temperature controlled Tecan Infinite (San Jose, Calif.) 200 M plate reading spectrometer at 37° C. For example, each experimental implementation was run with an initial 50 μL sample volume with a spring concentration of 100 nM in black 96 well plates. The exemplary plates were covered with a sticky film covers instead of the traditional clear plastic plate cover, e.g., because they reduced the error in measurements caused by evaporation. Addition of the extending or contracting strands in-between cycles may yield about 30 seconds of error in the measurements, e.g., because of the time required to add the strands and restart the machine. The successive extension and contraction cycles of the zipper springs were performed as follows. For example, the first extension and contraction cycle was performed by adding 10 times more extending strands and 20 times more contracting strands than springs. The second extension and contraction cycles were performed by adding 30 times more extending strands and 40 times more contracting strands than springs. The final extension of the zipper springs was performed by adding 50 times more extending strands than springs. For each exemplary cycle, 1 μL of the appropriate extending or contracting strand was added. Exemplary internal controls were included in each plate to monitor intensity shifts from removing and reinserting the plate, evaporation, photo bleaching and dilution from the additional volumes. For example, appropriate slight corrections to the data plots were performed to correct for variations from these effects. The exemplary values including average values and standard errors were calculated using Microsoft Excel, and the average values were plotted and a trend line was added when appropriate.

Thermally annealed zippers self-assembled into their lowest energy configuration. For example, a custom cycling program was run in a PCR thermocycler (Mastercycler Personal, Eppendorf, Westbury, N.Y.) to accomplish this. The solution temperature was quickly raised to 94° C., beyond the double strand melting temperature, followed by a slow, controlled, cooling at a rate of 1° C. every 2 min. to a final temperature of 4° C.

Exemplary implementations were performed to demonstrate tunability of the extension and contraction functionalities of the disclosed zipper springs. For example, the kinetics of extension and contraction can be tuned, e.g., using two different toehold schemes. For example, a first scheme used single stranded toeholds with 6 nt built into the S_{N} side of the springs. These were positioned between the B and A_{N} sections and fluorescent labels were placed on B_{O}(IbFQ) and L_{O}(FAM)) strands. The exemplary 6 nt extending strands (SD_{E+6}) were created by placing a complementary 6 nt toehold into the S_{E} sequence. The 6 nt toeholds on the extending strands hybridized to the 6 nt toehold on the exemplary zipper springs. Likewise, subsequent contraction of the spring was performed with S_{C1} and S_{C2+6} (e.g., fitted with an appropriately placed a 6 nt complementary section). Also, for example, the two arms of the zipper spring were modified to accommodate the 12 nt toehold, which included for example, 6 nt being removed from B_{O}(IbFQ) creating Bo-6(IbFQ) and 6 nt being added to L_{O}(FAM) creating, L_{O+6}(FAM), respectively.

Exemplary sequences of the nucleotide units used in exemplary implementations are shown in Table 5 and Table 6. Estimated energies of interaction for exemplary extending and contracting reactions performed in exemplary implementations are presented Table 7.

Table 5 shows the exemplary DNA zipper sequences for nucleotide units of strands used in exemplary implementations of the disclosed DNA based zipper springs technology. Nucleotide sequences that are included in the exemplary hinge members are represented in white text and highlighted in black. Nucleotide sequences that are included in the exemplary arm members are in black text and highlighted in gray. Nucleotide sequences that are included in the exemplary linking toehold members (e.g., toeholds used for fast extension on the zipper springs) are represented in lower case text.

TABLE 5
Sequences for DNA springs

| | SEQ | |
|---|---|---|
| | ID | |
| | NO | |
| S_{W} | 24 | 5′-GCC ATA GTT AGA GCA TGC |
| | | GCC ATA GTI ITT TTI TTT ITT |
| | | ITT IIT TTI ITT TIT TIT IIT |
| | | TIT Itc ttt tCC GAA TGC AGC |
| | | TGC CAT TCC GAA TGC-3′ |
| S_{N} | 25 | 5′-CGC AAT CCA CCG ATC ATC |
| | | CGC AAT CC aaa tct CCC AAC |
| | | CAC AAC AAA CCA AAC CAA CAA |
| | | CAA ACA ACA CCA CTA TGG CGC |
| | | ATG CTC TAA CTA TGG C-3′ |
| S_{W} | 26 | 5′-GCC ATA GTT AGA GCA TGC |
| w/out I's | | GCC ATA GTG GTG TTG TTT GTT |
| | | GTT GGT TTG GTT TGT TGT GGT |
| | | TGG Gtc ttt tCC GAA TGC AGC |
| | | TGC CAT TCC GAA TGC-3′ |
| L_{O} | 27 | 5′-GCA TTC GGA ATG GCA GCT |
| | | GCA TTC GG/FAM-3′ |
| L_{O + 6} | 28 | 5′-GCA TTC GGA ATG GCA GCT |
| | | GCA TTC GGA AAA GA/FAM-3′ |
| B_{O} | 29 | 5′-IbFQ/GGA TTG CGG ATG ATC |
| | | GGT GGA TTG CG-3′ |
| B_{W} | 30 | 5′-Cy3/IIA TTI CII ATI ATC |
| | | IIT IIA TTI CI/Cy5-3′ |
| B_{O} | 31 | 5′-GGA TTG CGG ATG ATC GGT |
| (used in | | GGA TTG CG-3′ |
| gels) | | |
| B_{O - 6} | 32 | 5′-IbFQ/CGG ATG ATC GGT GGA |
| | | TTG CG-3′ |
| S_{E} | 33 | 5′-AGA AGT AAG TAG GGT GTT |
| | | GTT TGT TGT TGG TTT GGT TTG |
| | | TTG TGG TTG GGA AGT GAG CGT |
| | | AA-3′ |
| S_{E}(Cy5) | 34 | 5′-/5Cy5/AGA AGT AAG TAG GGT |
| | | GTT GTT TGT TGT TGG TTT GGT |
| | | TTG TTG TGG TTG GGA AGT GAG |
| | | CGT AA-3′ |
| S_{C1} | 35 | 5′-ACA ACA AAC AAC ACC CTA |
| | | CTT ACT TCT-3′ |
| S_{C1}(IbRQ) | 36 | 5′-ACA ACA AAC AAC ACC CTA |
| | | CTT ACT TCT/3IbRQ-3′ |
| S_{C2} | 37 | 5′-TTA CGC TCA CTT CCC AAC |
| | | CAC AAC AAA-3′ |
| S_{E + 6} | 38 | 5′-GGT GTT GTT TGT TGT TGG |
| | | TTT GGT TTG TTG TGG TTG GG |
| | | aga ttt A AGT GAG CGT AA-3′ |
| S_{E + 6} | 39 | 5′-5IAbFQ/TTA CGC TCA CTT |
| (IbFQ) | | aaa tct CCC AAC CAC AAC AAA |
| | | CCA-3′ |
| S_{C + 6} | 40 | 5′-TTA CGC TCA CTT aaa tct |
| | | CCC AAC CAC AAC AAA CCA-3′ |
| S_{C + 6}(FAM) | 41 | 5′-GGT GTT GTT TGT TGT TGG |
| | | TTT GGT TTG TTG TGG TTG GG |
| | | aga ttt A AGT GAG CGT AA/36- |
| | | FAM-3′ |
| SD_{E + 6} | 42 | 5′-AGA AGT AAG TAG GGT GTT |
| | | GTT TGT TGT TGG TTT GGT TTG |
| | | TTG TGG TTG GG aga ttt A AGT |
| | | GAG CGT AA-3′ |
| S_{C2 + 6} | 43 | 5′-TTA CGC TCA CTT aaa tct |
| | | CCC AAC CAC AAC AAA-3′ |
| S_{E + 12} | 44 | 5′-AGA AGT AAG TAG GGT GTT |
| | | GTT TGT TGT TGG TTT GGT TTG |
| | | TTG TGG TTG GG aga ttt gga |
| | | ttg A AGT GAG CGT AA-3′ |
| S_{C + 12} | 45 | 5′-TTA CGC TCA CTT caa tcc |
| | | aaa tct CCC AAC CAC AAC AAA |
| | | CCA-3′ |
| S_{C2 + 12} | 46 | 5′-TTA CGC TCA CTT caa tcc |
| | | aaa tct CCC AAC CAC AAC |
| | | AAA-3′ |
| S_{E}0I | 47 | 5′-GGT GTT GTT TGT TGT TGG |
| | | TTT GGT TTG TTG TGG TTG GG-3′ |
| S_{E}5I | 48 | 5′-IGT GTT GTT TIT TGT TGG |
| | | TTT IGT TTI TTG TGG TTG IG-3′ |
| S_{E}7I | 49 | 5′-IGT GTT ITT TGT TIT TGG |
| | | TTT IGT TTG TTI TGG TTI IG-3′ |
| S_{E}9I | 50 | 5′-IGT ITT GTT TIT TGT TIG |
| | | TTT IGT TTI TTG TIG TTI GI-3′ |
| S_{E}13I | 51 | 5′-GIT ITT ITT TGT TIT TII |
| | | TTT GIT TTI TTI TII TTI GI |
| | | AAG TGA-3′ |
| S_{E}17I | 52 | 5′-ITT ITT ITT TIT TIT TII |
| | | TTT ITT TTI TTI TII TTI II-3′ |

Table 6 shows the exemplary DNA zipper sequences for nucleotide units of strands used in exemplary implementations of the disclosed DNA based zipper springs technology.

TABLE 6
DNA sequences for A and B zippers

| | SEQ | |
|---|---|---|
| | ID NO | |
| A_{W} | 53 | 5′-FAM/IIT ITT ITT TIT TIT TII TTT |
| | | IIT TTI TTI TIT TTI II/Cy5-3′ |
| A_{N} | 54 | 5′-IbRQ/CCC AAC CAC AAC AAA CCA AAC |
| | | CAA CAA CAA ACA ACA CC/IbFQ-3′ |
| A_{O} | 55 | 5′-GGT GTT GTT TGT TGT TGG TTT GGT |
| | | TTG TTG TGG TTG GG-3′ |
| B_{W} | 56 | 5′-Cy3/IIA TTI CII ATI ATC IIT IIA |
| | | TTI CI/Cy5-3′ |
| B_{N} | 57 | 5′-IbRQ/CGC AAT CCA CCG ATC ATC CGC |
| | | AAT CC/IbFQ-3′ |
| B_{O} | 58 | 5′-GGA TTG CGG ATG ATC GGT GGA TTG |
| | | CG-3′ |

Table 7 shows the energy calculations of the transitions, e.g., assuming equal concentrations of all interacting strands with a 160 mM NaCl concentration. The presented ΔG₃₇ energy values can be representative of the actual usable energy of the interaction for which they were calculated. The energy calculations also take the helix formation energy of the incoming extending and contracting strands into account.

TABLE 7

| | | | |
|---|---|---|---|
| | | Enthalpy | Entropy |
| | Gibbs (ΔG₃₇) | (ΔH) | (ΔS) |
| Interacting components | [kJ/mol] | [kJ/mol] | [kJ/mol] |

DNA A zipper

| | | | |
|---|---|---|---|
| Holding A_{W} to A_{N} (A zipper closed) | −87.90 | −1211.79 | −3.6237 |
| Holding A_{O} to A_{N} (A zipper opened) | −179.32 | −1271.23 | −3.5206 |
| Favoring the A zipper opening reaction | −91.42 | | |

DNA springs opened by S_{E} and closed by S_{C1} and S_{C2}

| | | | |
|---|---|---|---|
| Holding the springs contracted | −87.90 | −1211.79 | −3.6237 |
| Holding S_{E} to A_{N} (extended springs) | −179.32 | −1271.23 | −3.5206 |
| Favoring S_{E} to extend the springs | −91.42 | | |
| Holding S_{C1} to S_{E} | −112.39 | −859.67 | −2.4134 |
| Holding S_{C2} to S_{E} | −124.57 | −882.78 | −2.4447 |
| Favoring S_{C1} to hybridizes to S_{E} | −50.81 | | |
| Favoring S_{C2} to hybridizes to S_{E} | −60.11 | | |
| Favoring S_{C1} and S_{C2} to hybridize to S_{E} | −110.88 | | |
| Favoring the springs to contract after the extending strand is | −87.90 | | |
| removed by the contracting strands | | | |

DNA springs extended by S_{E+6} and contracted by S_{C1+6} and S_{C2}

| | | | |
|---|---|---|---|
| Holding S_{E+6} to A_{N} plus the 6 nt toehold on the springs | −202.21 | −1459.59 | −4.0541 |
| Favoring S_{E+6} to extend the springs | −114.31 | | |
| Holding S_{C+6} to S_{E+6} | −162.74 | −1180.40 | −3.2812 |
| Favoring S_{C+6} to hybridize to S_{E+6} | −50.81 | | |

DNA springs opened by SD_{C+6} and closed by S_{C+6}

| | | | |
|---|---|---|---|
| Holding SD_{C+6} to A_{N} plus the 6 nt toehold on the springs | −202.21 | −1459.59 | −4.0541 |
| Favoring SD_{E+6} to extend the springs | −114.31 | | |
| Holding S_{C1+6} to SD_{E+6} | −144.62 | −1076.17 | −3.0036 |
| Holding S_{C2} to SD_{E+6} | −124.57 | −882.78 | −2.4447 |
| Favoring S_{C1+6} and S_{C2} to hybridize to S_{E+6} | −110.88 | | |

DNA springs extended by S_{E+12} and contracted by S_{C+12} or S_{C1+12} and S_{C2}

| | | | |
|---|---|---|---|
| Holding S_{E+12} to A_{N} and the 12 nt toehold on the springs | −231.47 | −1670.97 | −4.6413 |
| Favoring S_{E+12} to extended the springs | −143.57 | | |
| Holding S_{C+12} to S_{E+12} | −194.89 | −1386.75 | −3.8429 |
| Favoring S_{C+12} to hybridize to S_{E+12} | −50.81 | | |
| Holding S_{C1+12} to S_{E+12} | −176.72 | −1282.53 | −3.5653 |
| Holding S_{C2} to S_{E+12} | −124.57 | −882.78 | −2.4447 |
| Favoring S_{C1+12} and S_{C2} to hybridize to S_{E+12} | −110.88 | | |

DNA springs extended by extending strands with various G bases substituted by I

| | | | |
|---|---|---|---|
| Holding S_{E}0I to the A_{N} portion of the springs | −179.32 | −1271.23 | −3.5206 |
| Favoring S_{E}0I to extend the contracted springs | −91.42 | | |
| Holding S_{E}5I to the A_{N} portion of the springs | −148.09 | −1240.67 | −3.5227 |
| Favoring S_{E}5I to extend the contracted springs | −60.19 | | |
| Holding S_{E}7I to the A_{N} portion of the springs | −137.00 | −1231.04 | −3.5275 |
| Favoring S_{E}7I to extend the contracted springs | −49.10 | | |
| Holding S_{E}9I to the A_{N} portion of the springs | −123.10 | −1201.32 | −3.4765 |
| Favoring S_{E}9I to extend the contracted springs | −35.20 | | |
| Holding S_{E}13I to the A_{N} portion of the springs | −102.97 | −1196.30 | −3.5251 |
| Favoring S_{E}13I to extend the contracted springs | −15.07 | | |
| Holding S_{E}17I to the A_{N} portion of the springs | −87.90 | −1211.79 | −3.6237 |
| Favoring S_{E}17I to extend the contracted springs | 0 | | |

DNA B zipper

| | | | |
|---|---|---|---|
| Holding B zipper closed | −63.79 | −703.21 | −2.0617 |
| Holding B zipper open | −133.44 | −884.04 | −2.4201 |
| Favoring the B zipper opening reaction | −69.65 | | |

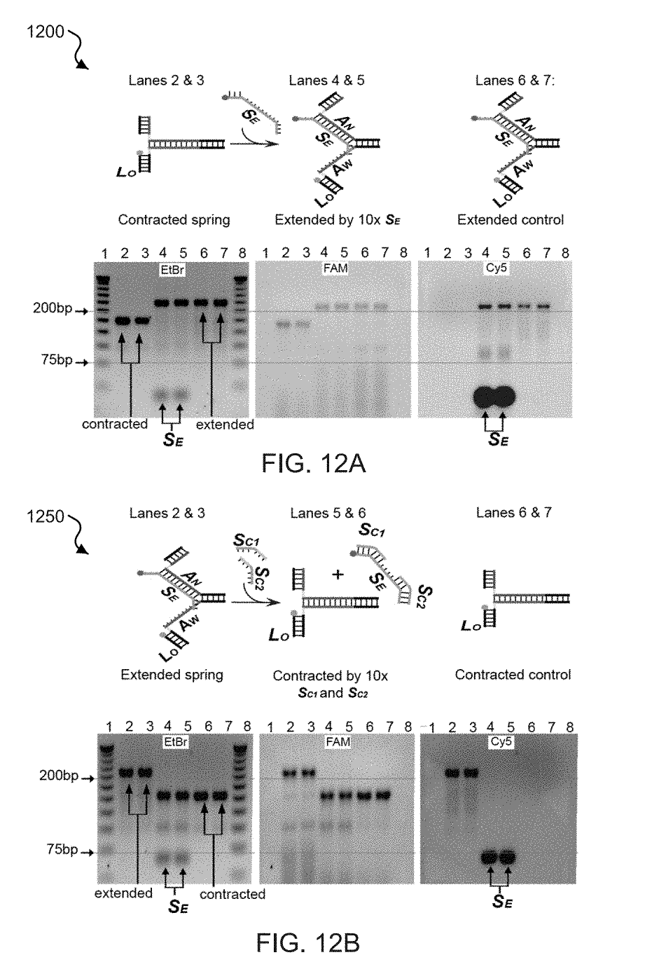

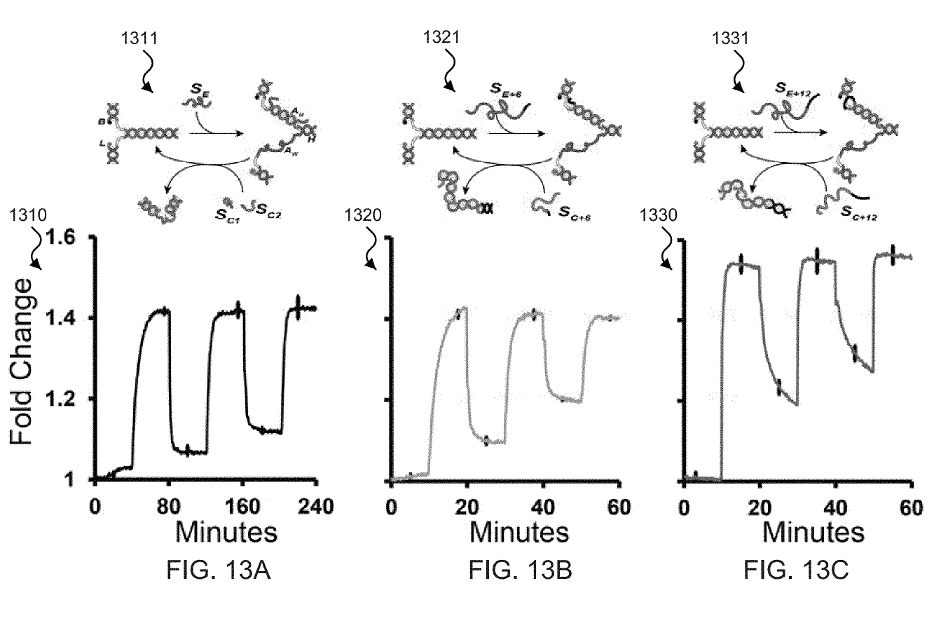

Exemplary implementations of the disclosed molecular zipper based springs were performed to examine the functionality of the zipper spring, e.g., with several different extension and contraction strands. For example, the reversible actuation of the zipper springs was visualized through gel electrophoresis (as shown in FIGS. 12A and 12B) and time-lapsed fluorescence (as shown in FIGS. 13A-C).

FIGS. 12A and 12B show fluorescent DNA gel electrophoresis data of the transitions exhibited by the exemplary zipper springs. Fluorescence images of EtBr, FAM and Cy5 were independently captured and displayed side-by-side. FIG. 12A shows fluorescent DNA gel electrophoresis data plots 1200 and corresponding schematic illustrations of the extension transition exhibited by exemplary contracted zipper springs. A 25 bp DNA ladder is visible in the EtBr images, e.g., shown in lanes 1 and 8. Lanes 4 and 5 contain zipper springs extended from the addition of 10 times S_{E}(Cy5) to the closed springs. Excess S_{E}(Cy5) is shown at approximately the 62 base pair (bp) position in the Cy5 image. For comparison, the FAM labeled contracted springs (lanes 2 and 3) and springs extended by S_{E}(Cy5) shown in lanes 6 and 7 are included. FIG. 12B shows fluorescent DNA gel electrophoresis data plots 1250 and corresponding schematic illustrations of the contraction transition exhibited by exemplary extended zipper springs. For example, contraction of the extended zipper springs were implemented with an equal concentration of S_{E}(Cy5) (e.g., assembled by thermal annealing). A 25 bp DNA ladder is visible in the EtBr images, e.g., shown in lanes 1 and 8. Lanes 4 and 5 contain the zipper springs contracted by adding 10 times more S_{C1} and S_{C2} to springs extended by S_{E}(Cy5). The removed S_{E}(Cy5) is at approximately the 62 bp position in the Cy5 image. For comparison, the zipper springs extended by S_{E}(Cy5) (e.g., shown in lanes 2 and 3) and the FAM labeled contracted springs (e.g., shown in lanes 6 and 7) and are included.

FIGS. 13A-13C show time-lapse fluorescence signal plots and corresponding illustrative schematics for the exemplary zipper springs reactions at 37° C. FIG. 13A shows a time-lapse fluorescence signal plot 1310 and a corresponding schematic illustration 1311 of an exemplary zipper spring device undergoing successive extension and contraction cycles with exemplary S_{E} extending strands and exemplary S_{C1} and S_{C2} contracting strands. For example, when the zipper springs are contracted, the fluorescent reporters are co-localized giving a minimum in the fluorescence. Likewise, when the zipper springs are extended the fluorescence is at a maximum. As shown in the plot 1310, initially, the exemplary zipper springs were contracted (0-40 min). The exemplary zipper springs were extended, e.g., by the addition of 10 times more S_{E} strands than zipper springs (40-80 min), and then contracted, e.g., by the addition of 20 times more S_{C1} and S_{C2} strands (80-120 min). The second extension and contraction cycle used 30 times the S_{E} strands (120-160 min), and 40 times the S_{C1} and S_{C2} strands, respectively, followed by 50 times the S_{E} strands (200-240 min). FIG. 13B shows a time-lapse fluorescence signal plot 1320 and a corresponding schematic illustration 1321 of an exemplary zipper spring device undergoing successive extension and contraction using exemplary S_{E+6} extending strands (e.g., an extending strand configured with a 6 nt toehold) and exemplary S_{C+6} contracting strands (e.g., a long single contracting strand configured with a 6 nt toehold). FIG. 13C shows a time-lapse fluorescence signal plot 1330 and a corresponding schematic illustration 1331 of an exemplary zipper spring device undergoing successive extension and contraction using exemplary S_{E+12} strands (e.g., an extending strand configured with a 12 nt toehold) and exemplary S_{C+12} strands (e.g., a long single contracting strand configured with a 12 nt toehold). Exemplary error bars shown in the plots 1310, 1320, and 1330 represent the standard error from three successive implementations.

For example, the zipper springs were monitored by tagging the inward facing ends of an L strand and a B strand with a fluorescent reporter (FAM) and quencher (IbFQ), respectively. For example, when the two fluorophores co-localized, the zipper springs contracted and quenched the fluorescence (as seen in the plot 1310 in FIG. 13A, [0-40 min]). For example, when the zipper springs were in the extended position, the separation between the reporters and quenchers was increased, e.g., resulting in an increase in fluorescence. Almost immediately after the addition of the S_{E} extending strands to the contracted springs, a sharp increase in the fluorescence intensity was observed (as seen in the plot 1310 in FIG. 13A, [40-80 min]). This exemplary fluorescence intensity dropped upon addition of the S_{C1} and S_{C2} contracting strands (as seen in the plot 1310 in FIG. 13A, [80-120 min]). The exemplary zipper springs were able to undergo multiple extension/contraction cycles, e.g., by adding successively higher concentrations of the extending and contracting fuel strands. The kinetic reaction rate constants for all of the exemplary reactions were found by curve fitting the fluorescence data and are presented in Table 9, presented later in the patent document.

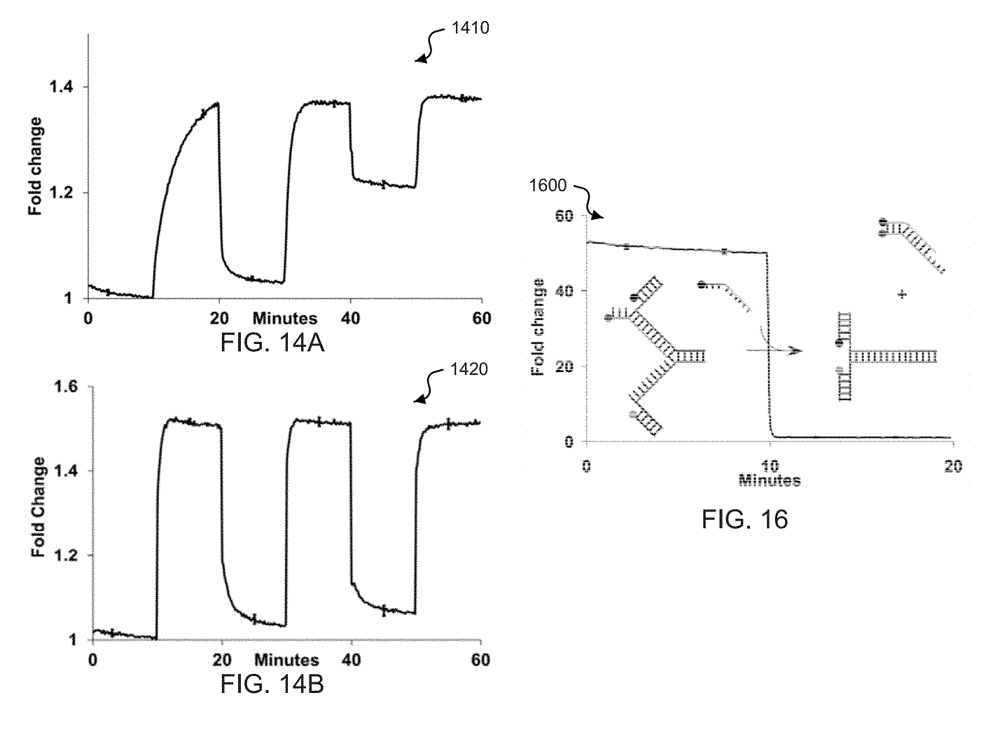

For example, the extension rate for the zipper springs was sped up by extending one of the exemplary toeholds on the S_{E} strand by an extra 6 nt or 12 nt (e.g., the S_{E+6} or S_{E+12} strands shown in illustrations 1321 and 1331, respectively). These exemplary extra sequences were complementary to the toehold built into the zipper springs between its A_{N} and B sections (as shown in the illustration 1120 of FIG. 11B). The addition of the exemplary toeholds into the extending strands can significantly increase the extending kinetics of the zipper springs, e.g., because of the rapid hybridization rate of single-stranded DNA. It can also significantly increase the amount of free energy favoring the extending reaction (as shown in Table 7). For example, the zipper springs were contracted using single contracting strands (e.g., the S_{C+6} or S_{C+12} strands) after extension with the S_{E+6} or S_{E+12} strands, as shown in FIGS. 13B and 13C. Successive extension and contraction cycles using a set of two different contracting strands with a 6 nt (S_{C1+6} and S_{C2}) or 12 nt (S_{C1+12} and S_{C2}) toehold are also included in FIGS. 14A and 14B.

FIGS. 14A and 14B show time-lapse fluorescence spectra plots from successive extension and contraction cycles of exemplary zipper springs at 37° C. As shown in a plot 1410 of FIG. 14A, initially, the zipper springs were contracted (0-10 min) followed by successive extension and contraction using a SD_{E+6} strand (e.g., 6 nt toehold extending strand) and S_{C1} and S_{C2+6} strands (e.g., two contracting strands). As shown in plot 1420 of FIG. 14B, the zipper springs were initially contracted followed by successive extension and contraction using a S_{E+12} strand (e.g., 12 nt toehold extending strand) and S_{C1} and S_{C2+12} strands (e.g., two contracting strands). Exemplary error bars in the plots represent the standard error from three successive implementations.

Exemplary implementations were performed to examine the hybridization rate of single closing strands compared to the closing rate of an exemplary zipper spring. Small exemplary DNA hairpins have been shown to re-hybridize closed in a few milliseconds once disassociated. This was investigated by placing a fluorescent reporter on S_{E+6}(FAM) and a quencher on S_{C+6}(IbFQ). Experimentally, this observes the hybridization rate of S_{C+6}(IbFQ) with S_{E+6}(FAM) which should be relatively close to the spring's contraction reaction. Their hybridization rate was found to be k=7.9±3.3×10⁴M⁻¹ s⁻¹. Comparison of this rate constant with that of the contracting spring (k=1.7+0.3×10⁴ M⁻¹ s⁻¹) suggests that the contracting rate of the spring is mostly dominated by the rate at which the extending strand is removed.

The specificity of the contracting strands can be further enhanced by increasing the length of the contracting strands and by incorporating a small zipper duplex into the toehold of the extending strands. For example, for the contracting strand to hybridize with the toehold on the extending strand, it can first displace the zipper and then remove the extending strand. These exemplary modifications can increase the specificity to the contracting strands, but may also slow down the kinetics.

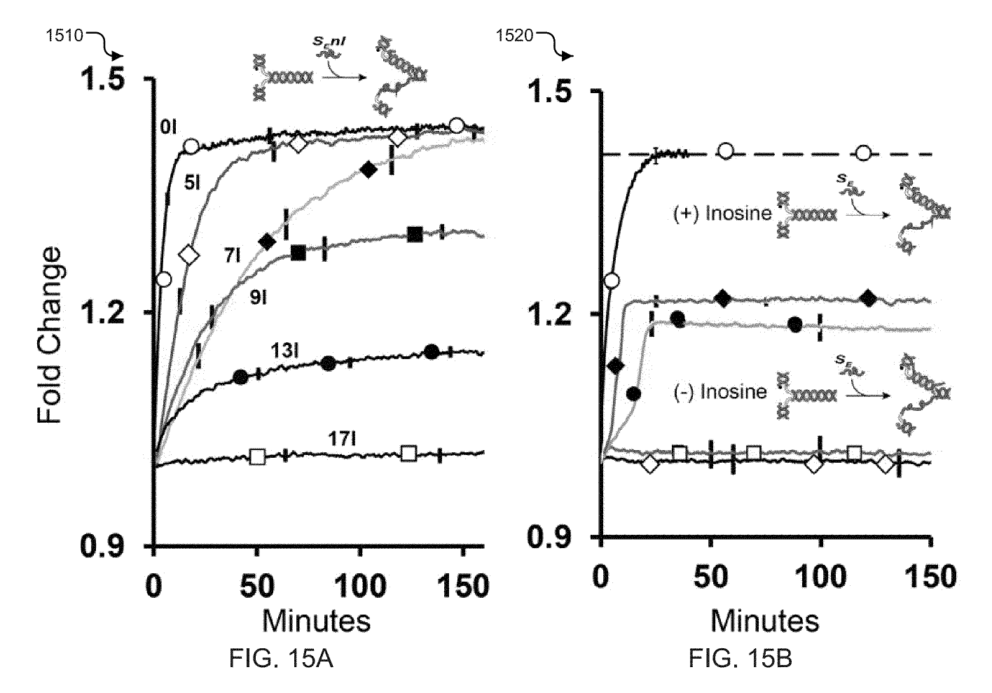

FIGS. 15A and 15B show time-lapse fluorescence signal plots for the exemplary zipper springs' extension with inosine-containing extending strands (plot 1510 of FIG. 15A) and using a zipper-less spring configuration (plot 1520 of FIG. 15B) at 37° C. For example, replacing guanine in the extending strands with inosine can reduce the energy driving the extension reaction of the zipper springs. As shown in the plot 1510, decreased extension kinetic rates and incomplete reactions were observed using extending strands S_{E}nI containing n=5 inosines (5I), 7 inosines (7I), 9 inosines (9I), 13 inosines (13I), and 17 inosines (17I). The exemplary results from adding 10 times more S_{E}nI extending strands are shown in plot 1510 as S_{E}5I (⋄), S_{E}7I (♦), S_{E}9I (▪), and S_{E}13I (●), which are plotted together with S_{E}0I (◯) and S_{E}17I (□) for comparison. As shown in the plot 1520, exemplary zipper springs configured without the inosine containing zipper mechanism were extended using 100 times S_{E} (⋄), 100 times S_{E+6} (□), 1600 times S_{E} (●) and 1600 times S_{E+6}(♦). For comparison an inosine zipper extended with 10 times S_{E} (◯) is included. Exemplary error bars in the plots represents the standard error from three successive implementations.

For example, the extension rates of the zipper springs can be decreased by substituting inosine in the place of guanine in the extending strand sequence (as shown in Table 5). For example, this decreased the driving energy of the zipper mechanism by ΔH≈8 kJ/mol for each inosine included in the extending strands. In this example, the weak side of the exemplary zipper sequence built into the zipper springs contained 17 inosines. The exemplary results in FIG. 15A showed the completeness of the extension reaction decreased with the diminishing energy of the extending strands. The extending reaction of the zipper springs using the complete zipper mechanism was shown to be relatively complete, e.g., which can be attributed to the increase in fluorescence from zipper springs extended using the S_{E} and S_{E+6} strands that was shown to be close to each other (also shown in Table 8).

Table 8 shows exemplary data of the extending controls of the spring. Exemplary zipper springs were extended with 10 times and 110 times more S_{E} strands and S_{E+6} strands than zipper springs. The similarities in the fold change of the different strands with different energies driving the extension reaction and the lack of change with increased extending strand concentrations suggests that the extension reactions using the full zipper mechanism are all relatively complete.

TABLE 8

| Opening Strand | 10 times more | 110 times more |
|---|---|---|
| S_{E} | 1.476657 | 1.476218 |
| S_{E+6} | 1.4477 | 1.483815 |

Exemplary implementations were performed to examine the contraction times of the exemplary zipper springs using a single contracting strand as compared to two separate contracting strands. For example, single contracting strands (S_{C+6}) and (S_{C+12}) closed the springs in about the same amount of time as their two-strand counterparts, but the use of a single contracting strand may increase the practicality of the exemplary zipper springs, e.g., by using a single DNA sequence to trigger the extension or contraction of the zipper springs.

The contraction rate of an individual zipper spring, after the extending strand is removed by the contracting strands, is on the order of a few milliseconds. This suggests that the contraction rate of the springs should mostly be dominated by the hybridization rate of the contracting strand with the extending strand. This was verified by placing a FAM fluorescent reporter on S_{E+6} and an IbFQ quencher on S_{C+6} shown in FIG. 16.

FIG. 16 shows a time-lapse fluorescence plot 1600 demonstrating the contraction function of exemplary zipper springs at 37° C. The springs were thermally annealed with an equal concentration of S_{E+6}(FAM) strands and contracted by addition of 10 times more S_{C+6}(IbFQ) strands than exemplary zipper springs. Once the S_{E+6}(FAM) strand holding the zipper springs extended was removed, the springs contracted within a few milliseconds. The almost spontaneous contraction of the springs is demonstrated by similar k-values for the two reactions. This exemplary implementation measured the rate at which the S_{C+6}(IbFQ) strand hybridizes to S_{E+6}(FAM) strand. The exemplary error bars represent the standard error from three successive implementations.

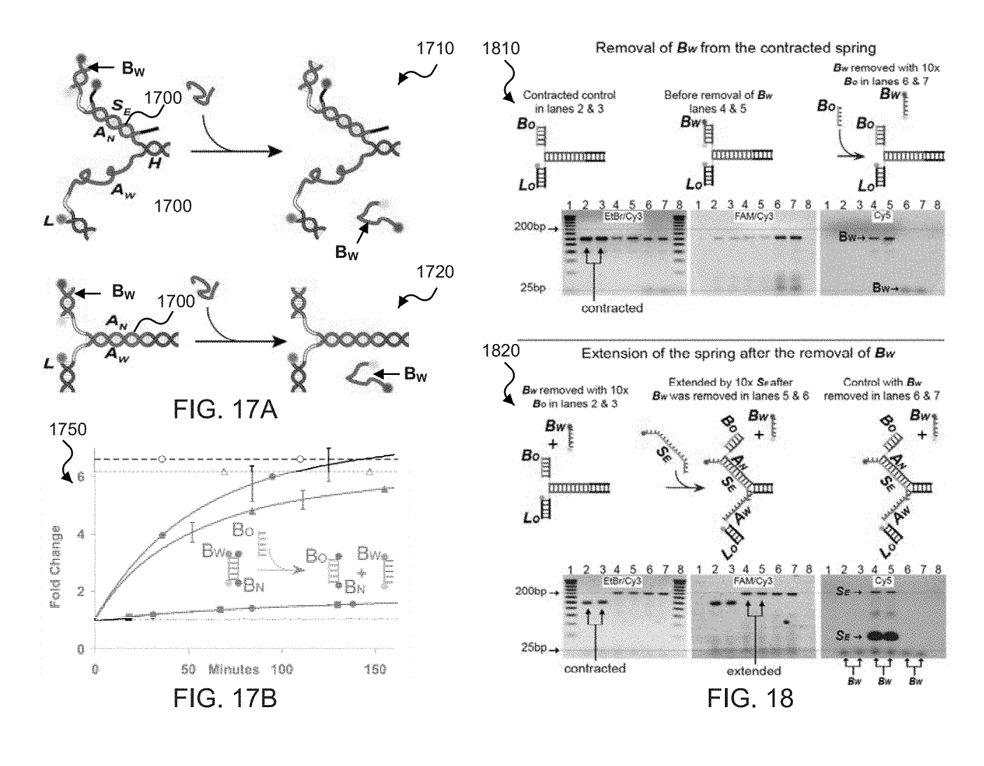

The disclosed zipper mechanism can be produced to be highly sequence specific, which can allow for more than one zipper to function independently within a single device. Exemplary implementations were performed to demonstrate the independence of functionality of the disclosed technology. For example, the B arm members of the zipper springs were transformed into a zipper by changing all of the guanines in its sequence to inosines (e.g., as shown in Table 6). This demonstrated the feasibility of incorporating multiple zipper or spring systems of the disclosed into a more elaborate device or system. For example, fluorescence analysis and gel electrophoresis data shown in FIGS. 17A, 17B, 18 and 19 demonstrate that the zipper arm was removed without affecting the function of the zipper spring.

The zipper spring mechanisms and the B arm members (e.g., which can also be configured to have zipper functionality) zipper actions can be configured to function independently from each other. Exemplary implementations were performed to demonstrate the functionality.

FIGS. 17A and 17B show illustrative schematics and time-lapse fluorescence measurement plots of exemplary zipper springs activity upon releasing an arm member. FIG. 17A shows a schematic illustration 1710 of the displacement of a B_{W} strand from an extended spring and a contracted spring 1700. FIG. 17A also shows a schematic illustration 1720 of a B_{W} strand removed independent of the extended and contracted states of the zipper spring 1700. FIG. 17B shows a plot 1750 of B zippers displacement reactions observed by tagging the ends of the B_{W} strand with a 3′Cy5 and 5′Cy3. For example, the addition of B_{O} resulted in a monotonically increasing fluorescence from both reporters indicating the separation of B_{W} from B_{N}3′Cy5 (●) and 5′Cy3 (▴). Upper dashed lines 3′Cy5 (◯) and 5′Cy3 (Δ) represent the fluorescence intensity of open reactions driven to completeness by thermal annealing. Lower dotted collinear lines are from the closed zippers prior to the reaction. The two lower collinear lines are the resulting fluorescence after addition of tenfold concentration of B_{N} without quenchers to the B zipper 3′Cy5 (▪) and 5′Cy3 (●). The exemplary error bars represent the standard error from three successive implementations.

FIG. 18 shows DNA gel determination data of the exemplary zipper springs from contracted to extended states. For example, a data panel 1810 shows gel data and corresponding illustrations of the independent removal of B_{W} from exemplary contracted zipper springs. As shown in the gel electrophoresis images, lanes 1 and 8 have a 25 bp DNA ladder and lanes 2 and 3 have the contracted zipper springs with FAM tagged to L_{O}. This exemplary result is confirmed with bands in the EtBr and FAM channels only. Lanes 4 and 5 have the contracted zipper springs with the tagged B_{W} as shown in the accompanying illustration and confirmed in EtBr, FAM and Cy5 channels. Lanes 6 and 7 have the contracted zipper springs with B_{W} displaced by B_{O}; this is shown in EtBr and FAM images collinear and single stranded B_{W} at ˜26 bp position in Cy5 channel. Also, for example, a data panel 1820 shows gel data and corresponding illustrations of the spring extension after removal of B_{W}. As shown in the gel electrophoresis images, lanes 1 and 8 have a 25 bp DNA ladder. The initially contracted zipper spring containing B_{O} are in lanes 2 and 3. The exemplary zipper spring is extended by adding a tenfold concentration of S_{E} is in lanes 4 and 5. The molecular weight increase observed in EtBr channels and the appearance of a collinear band in the Cy5 channel are demonstrative of S_{E} hybridizing to the springs and extending them. An extended spring assembled by thermal annealing and fitted with B_{O} and a 3′FAM fluorophore on L_{O} is included as a control in lanes 6 and 7.

For example, opening of an exemplary B arm member zipper is visualized with the exemplary B_{W} strand, e.g., used for time-lapse fluorescence measurements, e.g., B_{W} strand can be tagged with two fluorescent reporters (3′Cy5 and 5′Cy3). However, the Cy3 fluorophore cannot be visualized independently in the gel because of the spectral overlap between Cy3 and EtBr. The springs' extensions are performed with S_{E} and the contractions by S_{C1} and S_{C2}. For example, B_{W} can be removed by the opening strand B_{O}. The exemplary data in the data panels 1810 and 1820 demonstrate the stability, specificity and independent operation of the arm member zipper actions and the zipper spring actions.

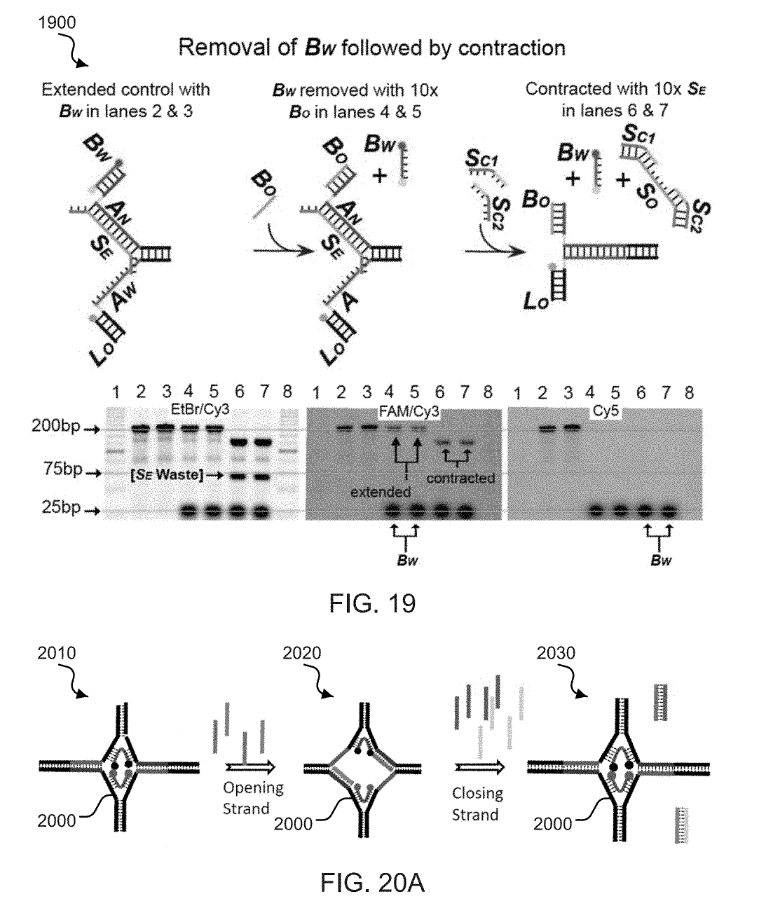

FIG. 19 shows a data panel 1900 including DNA gel determination data and corresponding illustrations of the exemplary zipper springs action after the removal of B_{W}. As shown in the data panel 1900, lanes 1 and 8 have 25 bp reference DNA ladders, and lanes 2 and 3 have extended springs with FAM tagged to L_{O}. Cy3 and Cy5 are tagged to B_{W}, so the extended zipper spring with B_{W} attached can be seen in all three channels. Lanes 4 and 5 have the extended spring with B_{W} removed, and thus the zipper spring in EtBr and FAM channels are visible collinearly. The single stranded B_{W} is seen at ˜26 bp position in the Cy5 channel and the EtBr and FAM channels because of the overlap of the Cy3 spectrum with EtBr and FAM. Lanes 6 and 7 have contracted springs with B_{W} removed, so the exemplary zipper spring presents in EtBr and FAM images collinearly and the single stranded B_{W} appears at ˜26 bp position in all three channels.

Exemplary calculations of kinetic rates of the exemplary DNA zipper springs are described. The rate constants (k) for the opening and closing of the DNA zipper springs were calculated in Matlab. The modeling was performed utilizing the function “lsqcurvefit” for least squares fitting of the parameters. For example, due to the stiff nature of the kinetics data and equations, integration of the differential equations was carried out using “ode23s”. For curve fitting, the data was scaled from 0 to 1 with 0 relating to the fully quenched state (e.g., all springs contracted) and 1 to maximum observed fluorescence when all the springs are extended.

The opening of the zipper springs from the contracted to the extended state was modeled as a second order reaction between the contracted spring (CS) and the extending strand (S_{E}) to produce a fluorescent extended spring (F) as represented by Eq. (6):

[S_{E}]+[CS]^{k}→[F] (6)

The standard second order kinetics equation was utilized for least squares fitting in Eq. (7):

$$\frac{d [ F ]}{dt} = k [ {S}_{E} ] [ CS ] \qquad ( 7 )$$

The concentration of extending strand ([S_{E}]) and contracted springs ([CS]) can be approximated utilizing the fluorescence data using the following relations in Eq. (8) and Eq. (9):

[CS]=1−[F] (8)

[S_{E}]=[S_{E}]₀−[F] (9)

where [S_{E}]₀ is the concentration of extending strand added to the reaction vessel.

When the spring extension did not run to completion (as determined by the fluorescence not reaching the maximum fluorescence observed when all strands are extended), the reaction was treated as being reversible. This was observed for the inosine substitution spring extension experiments. In this case, it was assumed that the weak portion (A_{W}) on the spring displaced the extending strand.

[S_{E}]+[CS]⇄[F]+[A_{W}] (10)

The concentration of the weak portion (A_{W}) was approximated by its local concentration (=160 μM=1600×). The kinetics equation then becomes:

$$\frac{d [ F ]}{dt} = {k}_{F} [ {S}_{E} ] [ CS ] - {k}_{R} [ F ] [ {A}_{w} ] \qquad ( 11 )$$

Closing of springs from extended to the contracted state was modeled as either a reversible second order or third order reaction depending on whether 2 or 1 contracting strands (S_{C}) were used to remove the extending strand from the spring device. The fluorescence decreases as a result of the addition of the contracting strands, however, adding excess contracting strands does not result in the contraction of all of the devices, e.g., indicating that removal of the S_{E} is a reversible process. The contracting strand was not able to extend the spring when added by itself at 100× concentrations to the contracted spring demonstrating a weak affinity to its compliment on the spring device. Thus, the closing was modeled as reversible reaction. The resulting equation becomes:

$$\frac{d [ F ]}{dt} = - {k}_{F} [ F ] [ {S}_{C} ] + {k}_{R} [ CS ] [ {S}_{E} {S}_{C} ] \qquad ( 12 )$$

In the models, it was assumed that free extending strands would bind quickly with free contracting strands reducing the effective concentration of the free contracting strands. The concentrations of the unbound and bound contracting strands were approximated as:

[S_{C}]=[S_{C}]₀[S_{E}] (13)

[S_{E}S_{C}]=[S_{E}] (14)

The amount of extending strand was calculated similarly when in excess of the contracting strand for the cycling implementations.

Table 9 shows the kinetics of the opening reaction with different constructs at 37° C. Reaction rate constants (k) together with their standard deviations (σₖ) and R²-value for the indicated zipper and spring reactions are shown.

TABLE 9
DNA springs cycled by successively increasing concentrations of the indicated extending and constricting
strands at the specified concentrations

| Spring reaction | 10 X | 20 X | 30 X |
|---|---|---|---|
| Extended by S_{E} and | S_{E} | S_{C1} and S_{C2} | S_{E} |
| contracted with S_{C1} | k = 2.1 ± 0.3 × 10³ M⁻¹ s⁻¹ | k_{F} = 8.4 ± 0.8 × 10⁶ M⁻² s⁻¹ | k = 4.0 ± 0.5 × 10³ M⁻¹ s⁻¹ |
| and S_{C2} | R² = 1.00 | k_{R} = 1.7 ± 0.4 × 10³ M⁻¹ s⁻¹ | R² = 0.99 |
| | | R² = 1.00 | |
| Extended by S_{E+6} and | S_{E+6} | S_{C+6} | S_{E+6} |
| contracted with S_{C+6} | k = 6.0 ± 0.4 × 10³ M⁻¹ s⁻¹ | k_{F} = 1.7 ± 0.3 × 10⁴ M⁻¹ s⁻¹ | k = 1.1 ± 0.7 × 10⁴ M⁻¹ s⁻¹ |
| | R² = 0.99 | k_{R} = 5.2 ± 1.2 × 10³ M⁻¹ s⁻¹ | R² = 1.00 |
| | | R² = 1.00 | |
| Extended by SD_{E+6} | SD_{E+6} | S_{C1+6} and S_{C2} | SD_{E+6} |
| and contracted with | k = 5.0 ± 0.1 × 10³ M⁻¹ s⁻¹ | k_{F} = 2.2 ± 0.2 × 10¹⁰ M⁻² s⁻¹ | k = 1.3 ± 0.2 × 10⁴ M⁻¹ s⁻¹ |
| S_{C1+6} and S_{C2} | R² = 1.00 | k_{R} = 2.1 ± 0.3 × 10³ M⁻¹ s⁻¹ | R² = 0.99 |
| | | R² = 1.00 | |
| Extended by S_{E+12} | SD_{E+12} | S_{C+12} | SD_{E+12} |
| and contracted with | k = 2.7 ± 0.2 × 10⁴ M⁻¹ s⁻¹ | k_{F} = 4.4 ± 0.5 × 10³ M⁻¹ s⁻¹ | k = 2.8 ± 0.2 × 10⁴ M⁻¹ s⁻¹ |
| S_{C+12} | R² = 0.97 | k_{R} = 2.5 ± 0.4 × 10³ M⁻¹ s⁻¹ | R² = 0.99 |
| | | R² = 1.00 | |
| Extended by S_{E+12} | S_{E+12} | S_{C1+12} and S_{C2} | S_{E+12} |
| and contracted with | k = 3.4 ± 0.2 × 10⁴ M⁻¹ s⁻¹ | k_{F} = 2.2 ± 0.4 × 10¹⁰ M⁻² s⁻¹ | k = 4.7 ± 0.3 × 10⁴ M⁻¹ s⁻¹ |
| S_{C1} and S_{C2} | R² = 1.00 | k_{R} = 2.3 ± 0.5 × 10³ M⁻¹ s⁻¹ | R² = 1.00 |
| | | R² = 1.00 | |

| Spring reaction | 40 X | 50 X |
|---|---|---|
| Extended by S_{E} and | S_{C1} and S_{C2} | S_{E} |
| contracted with S_{C1} | k_{F} = 3.6 ± 0.6 × 10⁹ M⁻² s⁻¹ | k = 5.2 ± 1.6 × 10³ M⁻¹ s⁻¹ |
| and S_{C2} | k_{R} = 9.0 ± 2.8 × 10² M⁻¹ s⁻¹ | R² = 1.00 |
| | R² = 0.98 | |
| Extended by S_{E+6} and | S_{C+6} | S_{E+6} |
| contracted with S_{C+6} | k_{F} = 7.4 ± 0.5 × 10³ M⁻¹ s⁻¹ | k = 1.1 ± 0.1 × 10⁴ M⁻¹ s⁻¹ |
| | k_{R} = 2.1 ± 0.2 × 10³ M⁻¹ s⁻¹ | R² = 0.99 |
| | R² = 0.96 | |
| Extended by SD_{E+6} | S_{C1+6} and S_{C2} | SD_{E+6} |
| and contracted with | k_{F} = 1.1 ± 0.1 × 10¹⁰ M⁻² s⁻¹ | k = 2.1 ± 0.5 × 10⁶ M⁻¹ s⁻¹ |
| S_{C1+6} and S_{C2} | k_{R} = 5.3 ± 1.1 × 10³ M⁻¹ s⁻¹ | R² = 1.00 |
| | R² = 0.97 | |
| Extended by S_{E+12} | S_{C+12} | SD_{E+12} |
| and contracted with | k_{F} = 3.2 ± 0.1 × 10³ M⁻¹ s⁻¹ | k = 6.0 ± 0.5 × 10⁴ M⁻¹ s⁻¹ |
| S_{C+12} | k_{R} = 1.1 ± 0.1 × 10³ M⁻¹ s⁻¹ | R² = 1.00 |
| | R² = 0.96 | |
| Extended by S_{E+12} | S_{C1+12} and S_{C2} | S_{E+12} |
| and contracted with | k_{F} = 3.6 ± 0.3 × 10¹⁰ M⁻² s⁻¹ | k = 3.6 ± 1.0 × 10⁴ M⁻¹ s⁻¹ |
| S_{C1} and S_{C2} | k_{R} = 2.1 ± 0.4 × 10³ M⁻¹ s⁻¹ | R² = 0.99 |
| | R² = 1.00 | |

DNA springs extended using extending strands with various G bases substituted by I

| Extension strand | 10 X |
|---|---|
| S_{E}0I (10 X) | k_{F} = 3.5 ± 0.2 × 10³ M⁻¹ s⁻¹ |
| | k_{R} = 3.6 ± 0.6 × 10⁰ M⁻¹ s⁻¹ |
| | R² = 0.97 |
| S_{E}5I (10 X) | k_{F} = 1.0 ± 0.1 × 10³ M⁻¹ s⁻¹ |
| | k_{R} = 3.4 ± 1.2 × 10⁻¹ M⁻¹ s⁻¹ |
| | R² = 1.00 |
| S_{E}7I (10 X) | k_{F} = 3.3 ± 0.1 × 10² M⁻¹ s⁻¹ |
| | k_{R} = 5.9 ± 6.2 × 10⁻² M⁻¹ s⁻¹ |
| | R² = 1.00 |
| S_{E}9I (10 X) | k_{F} = 3.9 ± 0.2 × 10² M⁻¹ s⁻¹ |
| | k_{R} = 1.5 ± 0.1 × 10⁰ M⁻¹ s⁻¹ |
| | R² = 1.00 |
| S_{E}13I (10 X) | k_{F} = 1.8 ± 0.1 × 10² M⁻¹ s⁻¹ |
| | k_{R} = 3.1 ± 0.3 × 10⁰ M⁻¹ s⁻¹ |
| | R² = 0.95 |
| S_{E}17I (10 X) | N/A |

Hybridization rate of S_{C1+6} (IbFQ) with S_{E} (Cy5) measured using springs assembled into the extended position
by thermal annealing the springs with a 1 X concentration of S_{E} (Cy5)

| Contraction strand | 10 X |
|---|---|
| S_{C1+6} (IbFQ) | k_{F} = 7.9 ± 3.3 × 10⁴ M⁻¹ s⁻¹ |
| | R² = 0.97 |

DNA zippers

| | 10X |
|---|---|
| A zipper [A_{W:}A_{N}] | |
| Opening strand A_{O} | k = 2.5 ± 1.6 × 10³ M⁻¹ s⁻¹ |
| | R² = 0.83 |
| B zipper [B_{W:}B_{N}] | |
| Opening strand B_{O} | k = 7.7 ± 4.5 × 10² M⁻¹ s⁻¹ |
| | R² = 0.94 |

The “local concentration” of a DNA zipper spring can be determined as the estimated bulk solution equivalent concentration of the two spring strands unhybridized. This exemplary value can describe the driving force for interaction that two co-localized strands have. In the exemplary calculations, a sequence of DNA can have a maximum interaction volume that is approximated by a sphere with the diameter equal to the length of the strand. For example, a 24 base pair (bp) DNA spring fully extended forms an isosceles right triangle with the hypotenuse that is 10.9 nm (e.g., assuming 0.32 nm/bp). A sphere with a 10.9 nm diameter has a volume of 671 nm³. For example, with one zipper spring contained within this volume, the local concentration of the zipper springs can be determined to be 2.47 mM. In other words, with all else being the same, the propensity for an assembled DNA spring to hybridize is equivalent to 2.47 mM of unhybridized DNA spring strands.

Exemplary implementations of the disclosed molecular zipper based springs can be employed to create composite devices. For example, to demonstrated this, the 26 nt B_{O} strand on the B arm of the springs was converted to a zipper by changing the 11 guanines in its sequence to inosines. This gives the springs a removable arm and could be chemically coupled to a surface or an object using a variety of functional groups, e.g., such as thiol modification, then unzipping B_{W} to release the objects from the springs (as exemplified in the illustration 1710 in FIG. 17A). Such a system could be useful in conditional activation situations, e.g., where a vehicle tethered to the B_{W} strand would be released upon specific recognition of the B_{O} opening strand. This exemplary method can be more robust than dangling single stranded toeholds in many applications because of the base pair specificity of the described zippers and their tunable kinetics. The specificity of the short zippers could also be further increased by incorporating locked nucleic acid (LNA) bases into the zipper springs. For example, LNA bases can increase the kinetics of the opening and closing of DNA zipper mechanisms.

The force created by the zippers can also be tuned by changing the base pair sequence of the zippers. For example, a strand including only C-G bonds requires a force of ˜20 pN to be torn apart, where as a strand solely composed of A-T bonds requires ˜9 pN, and a mixture of the bases is somewhere in-between these force values. The disclosed zipper mechanism of the zipper springs can be modified to contain C bases, and thereby tuning the force created by the zipper springs.

The disclosed molecular zipper based spring technology is compact, performs a defined contractile mechanical function, and can be implemented as an actuator (e.g., a motor to actuate DNA origami structures). The disclosed molecular zipper based spring technology includes tunable reaction kinetics with repeatable extension and contraction cycles. For example, exemplary DNA zipper springs demonstrate repeatable extension and contraction cycles and generate ˜9 pN of force during contraction, e.g., which is enough force to manipulate biological macromolecules. In addition, by changing the toehold length of an exemplary DNA zipper spring, the DNA zipper spring's extension and contraction duration can be tuned. Exemplary zipper springs of the disclosed technology can be useful in a variety of applications, e.g., including biomolecular interactions. For example, by using the exemplary zipper springs in dynamic DNA origami structures, these assemblies can become useful functional components in larger microfluidic lab-on-a-chip systems or in nanomedicine as part of a drug delivery system.

The exemplary DNA zipper tweezers and springs can be implemented as separate devices or on a single device, and these devices can be activated under specific environmental conditions, e.g., including temperature, pH, etc. For example, the DNA zipper-based tweezers and springs are self-regenerating, utilize longer fuel strands, and are reliably efficient (e.g., energetically self-sufficient, requiring no external energy, and preventing nonspecific binding of non-target molecules). Also, for example, the described zipper-based technology can provide flexibility in designing robust, compact and modular devices and systems that can be incorporated into multi-component and/or more elaborate DNA based nanomachines.

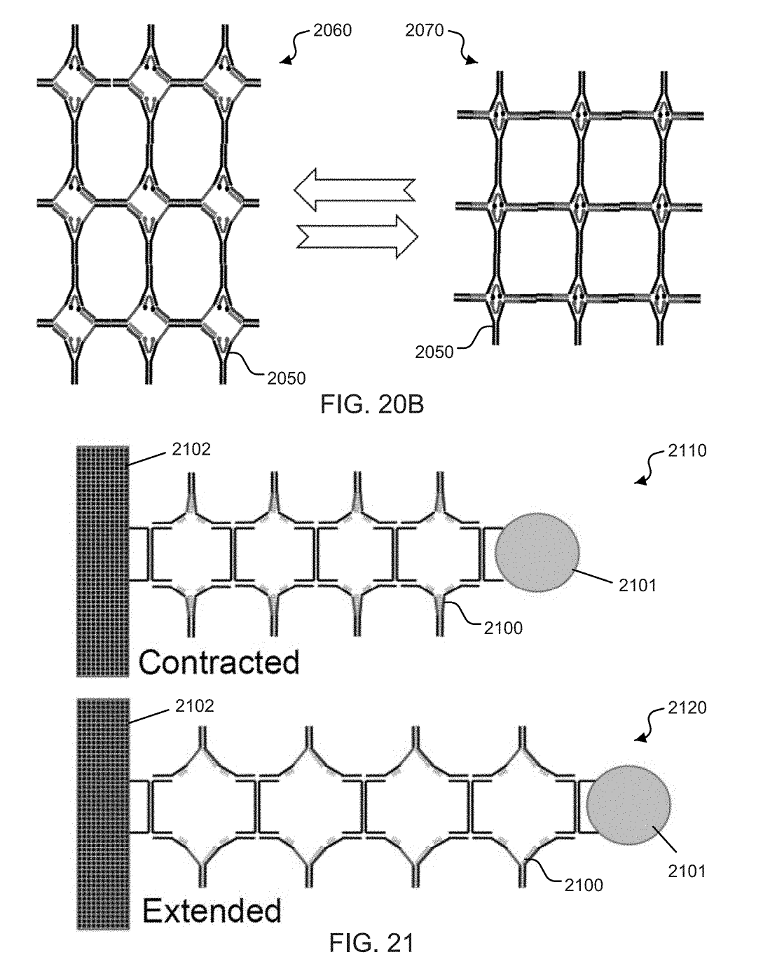

In another aspect, the disclosed technology can include engineering new structures and materials with the disclosed zipper constructs and integrating the disclosed zipper constructs with other materials, devices, systems, and techniques. For example, FIG. 20A shows an exemplary double zipper structure 2000 that includes the multiple structures employing the disclosed zipper mechanism that can be configured in a molecular zipper device. For example, the exemplary double zipper structure 2000 can be configured using nucleotide strands comprising naturally-occurring and non-naturally occurring nucleobases. FIG. 20A includes a panel 2010 that shows the double zipper structure 2000 in a contracted (e.g., zipped) position. A panel 2020 shows the double zipper structure 2000 in an extended (e.g., unzipped) position, e.g., by employing the disclosed zipper mechanism using an opening strand as previously described in this patent document. A panel 2030 shows the double zipper structure 2000 in a contracted (e.g., zipped) position, like that in the panel 2010, e.g., by employing the disclosed zipper mechanism using a closing strand as previously described in this patent document.

Various configurations of the disclosed molecular zipper can be engineered as structures that include multiple molecular zipper constructs, which can be implemented in nanoscale devices and systems. For example, the double zipper structure 2000 can be configured as a multiple zipper structure implemented in devices and systems that include array structures, position motors, gating elements, vehicles, and carriers.

FIG. 20B shows an exemplary array structure of DNA zipper mechanisms 2050 that is configured in a multidimensional sequences within the array. For example, the array 2050 can be configured in two or three dimensions. For example, the exemplary DNA zipper array can be implemented to change its size, thereby actuating a function, e.g., such as mechanical functions including motorization and gating. The exemplary array 2050 is shown in an opened (e.g., unzipped) position in the panel 2060, e.g., taking on a rectangle conformation. The exemplary array 2050 is shown in the contracted (e.g., zipped) position in the panel 2070, e.g., changing its shape to become a square conformation.

FIG. 21 shows an exemplary DNA zipper position motor 2100 that includes the disclosed zipper springs in a linear aligned arrangement. For example, the exemplary zipper motor 2100 can be configured as a two-state positioning motor, e.g., utilizing one type of zipper sequence that includes eight zipper strands, as shown in the figure. A panel 2110 shows the exemplary motor 2100 in the contracted position, and a panel 2120 shows the exemplary motor 2100 in the extended position. At least one structure 2101 (e.g., a micro-sized structure or nanoscale structure such as a nanoparticle, nanotube, etc.) and/or at least one substrate 2102 can be coupled to the motor 2100 that actuates the movement of the structure 2101.

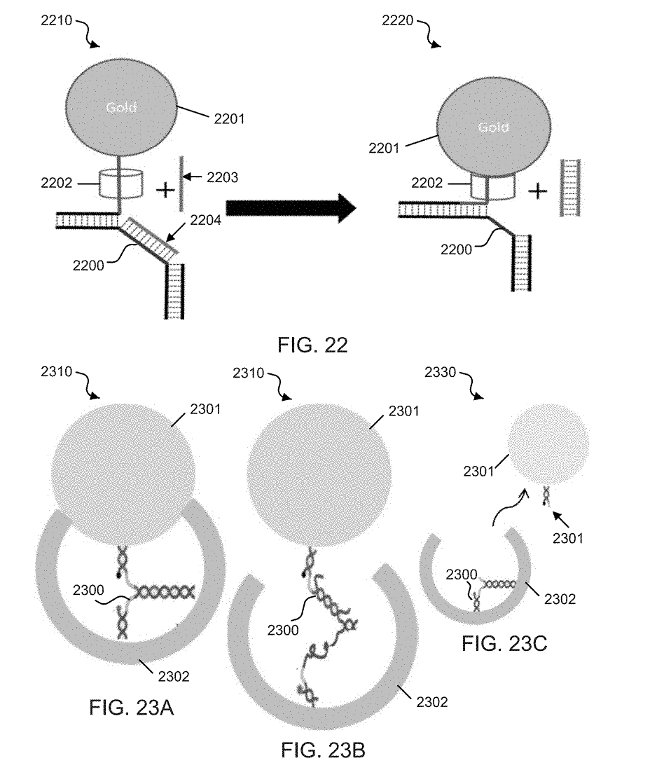

FIG. 22 shows an exemplary channel gating DNA zipper structure 2200 that includes an exemplary DNA zipper tweezers structure. For example, the zipper structure 2200 is shown in panel 2210 in an extended state, and thus a coupled particle 2201 (e.g., gold particle) is not completely blocking a channel 2202 (e.g., an ion channel). For example, upon introduction of an exemplary contraction strand 2203 (as shown in the panel 2210), the extension strand 2204 is removed and the zipper structure 2200 contracts (as shown in the panel 2220). This exemplary implementation of the zipper structure 2200 can be employed in a device for a variety of applications, e.g., using gold nanoparticles to plug the ion channels.

The disclosed molecular zipper technology can include controlled drug delivery devices, systems, and techniques using integrated nanocapsules with kinetically tunable lids employing the disclosed zipper mechanism. For example, exemplary controlled drug delivery devices can be implemented in a variety of applications, e.g., including biomedical applications such as using controlled release of biocompatible material to treat diseases and disorders. For example, an exemplary biodegradable nano-capsule with a movable lid of the disclosed technology can be implemented for long-term delivery of age-related macular degeneration (AMD) therapeutics, e.g., by controlling the lid opening/closing over an extended time and frequency using exemplary DNA zipper springs. For example, the DNA springs can include engineered nucleic acids constructs that allows tunable and regenerative motor and spring-like action. Other exemplary materials can be included within the exemplary controlled drug delivery device, e.g., including functionalized nanoparticles, imaging agents, enzymes, nucleic acids, or viral vectors, as well as other materials.

For example, intravitreal delivery of drugs and compounds can experience rapid clearance and hence require frequent injections. Controlled drug release over an extended period can reduce the frequency of these injections and allow on-demand release, e.g., for ocular diseases and disorders such as AMD but other diseases. The disclosed controlled drug delivery vehicles can include a degradable nanoscale container (e.g., a nanobowl or nanojar), an actuating molecular zipper construct, and a nanoscale degradable lid. The exemplary drug delivery vehicles can be configured to be biocompatible and immune protected.

For example, the degradable nanoscale container can be configured as a metal capsule or a hollow colloidal capsule. For example, gold can be used as initial plating material to create the hollow colloidal capsule, e.g., by evaporating gold onto polystyrene beads. The exemplary polystyrene beads can include biocompatible and biodegradable polymer materials, e.g., poly-1-lactic acid, poly(glycolic acid), and polycaprolactone. For example, the exemplary capsule can be coated with subsequent layers, e.g., by coating silica using the evaporation techniques.

FIGS. 23A-23C shows schematic illustrations of exemplary controlled drug delivery devices. For example, a controlled drug delivery device 2310 can include a self-splicing molecular zipper spring construct 2300 that can open a lid 2301 of an exemplary drug capsule 2302. The device 2310 is shown in FIG. 23A in a closed position, e.g., which can also include drugs or other materials and compounds contained within the capsule 2302. For example, therapeutic agents may be loaded by controlled drying of a solution containing the nanocapsules and the drug by itself, or suspended in a polymer emulsion or hydrogel. For example, as shown in FIGS. 23A-23C, the zipper spring construct 2300 can be configured as the disclosed DNA zipper based springs (e.g., the spring 1121 shown in FIG. 11B), e.g., including a self-splicing DNA sequence on the arms of the spring. For example, the zipper spring construct 2300 can include an exemplary nucleotide unit sequence that contains DNAzyme components that can cleave RNA. Exemplary DNAzyme components can be hair-pinned to the zipper spring construct 2300 (e.g., at room temperature), but can melt at body temperature (37° C.) and be free to cleave the target site. An exemplary DNA/RNA hybrid sequence can include the cleavage site on a complementary sequence near the DNAzyme. The exemplary zipper spring construct 2300 can be configured to be kinetically tunable. For example, by changing the number of self splicing strands that hold the capsule shut, the average opening time of the capsule can be changed. RNA cleavage rates can also be tuned by changing the nucleotide length around the active site of the DNAzyme and changing the active sequence of the DNAzyme. These two exemplary mechanisms can be implemented to adjust opening times, e.g., in a range between several minutes to several weeks. For example, the lid 2301 can comprise carboxylate-modified polymer materials to form the lid. Attachment of the zipper spring construct 2300 to the lid 2301 can be performed using amide linkers, or other linker chemistries, e.g., using a malemide-thiol bond.

FIG. 23B shows the device 2310 in an opened position, e.g., which can release drugs or other materials and compounds contained within the capsule 2302 to the environment in which the device 2310 is deployed. FIG. 23C shows an exemplary configuration of the device 2310 in which the zipper spring construct 2300 can release the lid 2301, e.g., by severing itself at a linking arm 2306 of the zipper spring construct 2300.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described above should not be understood as requiring such separation in all embodiments.

Only a few implementations and examples are described and other implementations, enhancements and variations can be made based on what is described and illustrated in this patent document.

1. A method of capturing a target molecule, comprising:
deploying a double-stranded molecule into a fluid environment, the double-stranded molecule including a binding strand having a sequence of nucleotides that is coupled to a passive strand having a complementary sequence of nucleotides; and
attaching a target molecule in the fluid environment to the binding strand, the target molecule including an opening strand having a complement sequence of nucleotides corresponding to the binding strand, wherein the attaching uncouples the passive strand as the nucleotides of the opening strand bond to the corresponding complement nucleotides of the binding strand.

2. The method of claim 1, wherein the fluid environment is within an organism.

3. The method of claim 1, wherein the attaching the target molecule to the binding strand includes the nucleotides of the opening strand forming a bond with the corresponding complement nucleotides of the binding strand at an energy greater than a bond between the passive strand and the binding strand.

4. The method of claim 1, wherein the attaching the target molecule to the binding strand includes detaching the passive strand from the double-stranded molecule.

5. The method of claim 1, wherein the attaching the target molecule to the binding strand uses no external energy.

6. The method of claim 1, wherein the opening strand includes less nucleotides than each of the binding strand and the passive strand.

7. The method of claim 6, wherein the attaching the target molecule to the binding strand does not detach the passive strand from the double-stranded molecule.

8. The method of claim 7, further comprising removing the target molecule from the double-stranded molecule by coupling the opening strand to a complement closing strand of a reset molecule.

9. The method of claim 8, further comprising recoupling the complementary sequence of nucleotides of the passive strand to the sequence of nucleotides of the binding strand, thereby regenerating the double-stranded molecule.

10. A method for controlled delivery of a payload, comprising:
deploying a nanocarrier device into a fluid environment, the nanocarrier device including:
a shell structure having a hollow interior and an opening that spans between the hollow interior and an exterior surface of the shell structure,
a double-stranded molecule including a binding strand and a passive strand, the binding strand having a sequence of nucleotides operable to bind to and unbind from a complementary sequence of nucleotides of the passive strand, wherein the double stranded molecule is attached to an interior surface within the hollow interior of the shell structure by one of the binding strand or the passive strand,
a nanoparticle attached to the other of the binding strand or the passive strand, wherein, when the sequence of nucleotides is bound to the passive strand, the nanoparticle seals the opening of the shell structure such that the nanocarrier device is closed, and when the sequence of nucleotides is unbound from the passive strand, the nanoparticle does not seal the opening of the shell structure such that the nanocarrier device is open,
wherein the deployed nanocarrier device is closed and loaded with a payload enclosed within the nanocarrier device; and
actuating the deployed nanocarrier device at a certain condition to cause the nanocarrier device to open and unseal the opening of the shell structure, wherein the certain condition includes one or more of a temperature or a time from sealing; and
releasing the payload out of the nanocarrier device into the fluid environment.

11. The method of claim 10, wherein the fluid environment is within an organism.

12. The method of claim 10, wherein the fluid environment is in an in vitro assay container.

13. The method of claim 10, further comprising:
loading the payload within the hollow interior of the shell structure of the nanocarrier device, wherein the nanocarrier device is open.

14. The method of claim 13, wherein the loading includes drying a solution containing the nanocarrier device and the payload over a controlled time period and temperature.

15. The method of claim 13, wherein the loading includes suspending the nanocarrier device and the payload in a polymer emulsion or hydrogel.

16. The method of claim 10, wherein the actuating the deployed nanocarrier device to open includes releasing a self-splicing nucleotide sequence from within the nanocarrier device to cleave nucleotide pairs of the binding strand and the passive strand.

17. The method of claim 16, wherein the self-splicing nucleotide sequence is released based on applied heat to the nanocarrier device.

18. The method of claim 17, wherein the self-splicing nucleotide sequence is released at a temperature of at least 37° C.

19. A nanocarrier device for controlled delivery of a payload, comprising:
a shell structure having a hollow interior and an opening that spans between the hollow interior and an exterior surface of the shell structure;
a double-stranded molecule including a binding strand and a passive strand, the binding strand having a sequence of nucleotides operable to bind to and unbind from a complementary sequence of nucleotides of the passive strand, wherein the double stranded molecule is attached to an interior surface within the hollow interior of the shell structure by one of the binding strand or the passive strand; and
a nanoparticle attached to the other of the binding strand or the passive strand, wherein, when the sequence of nucleotides is bound to the passive strand, the nanoparticle seals the opening of the shell structure such that the nanocarrier device is in a closed position, and when the sequence of nucleotides is unbound from the passive strand, the nanoparticle does not seal the opening of the shell structure such that the nanocarrier device is in an open position,
wherein the deployed nanocarrier device is loaded with a payload enclosed within the nanocarrier device in the closed position.

20. The nanocarrier device of claim 19, further comprising:
a first arm molecule, including a first binding hinge member having a first nucleotide sequence, and a first passive hinge member having a first complementary nucleotide sequence able to bind and unbind from the first nucleotide sequence of the first binding hinge member, wherein the first arm molecule is coupled to an end of the sequence of nucleotides of the binding strand; and
a second arm molecule, including a second binding hinge member having a second nucleotide sequence, and a second passive hinge member having a second complementary nucleotide sequence able to bind and unbind from the second nucleotide sequence of the second binding hinge member, wherein the second arm molecule is coupled to an end of the sequence of nucleotides of the passive strand.

21. The nanocarrier device of claim 20, further comprising:
a self-splicing nucleotide sequence on at least one of the first arm molecule or second arm molecule.

22. The nanocarrier device of claim 21, wherein the self-splicing nucleotide sequence includes a DNAzyme component operable to cleave nucleotide pairs of the binding strand and the passive strand when bound.

23. The nanocarrier device of claim 22, wherein the DNAzyme component is hair-pinned to the double-stranded molecule at room temperature and is operable to unpin from the double-stranded molecule at a temperature of at least 37° C.
